(19) United States
(12) Patent Application Publication
(10) Pub. No.: US 2026/0188464 A1
(43) Pub. Date: Jul. 2, 2026

(54) APPARATUS AND METHOD FOR PROVIDING PLATFORM-BASED GROWTH AND DIETARY MANAGEMENT SUPPORT SERVICES FOR CHILDREN AND ADOLESCENTS

(71) Applicant: INDUSTRY-ACADEMIC COOPERATION FOUNDATION, YONSEI UNIVERSITY, Seoul (KR)

(72) Inventors: Ji Won LEE, Seoul (KR); Hyung Mi KIM, Goyang-si (KR); Li Rang LIM, Seoul (KR); Jia LEE, Seoul (KR)

(73) Assignee: INDUSTRY-ACADEMIC COOPERATION FOUNDATION, YONSEI UNIVERSITY, Seoul (KR)

(21) Appl. No.: 19/433,661

(22) Filed: Dec. 26, 2025

(30) Foreign Application Priority Data
Dec. 31, 2024 (KR) 10-2024-0202950
Jul. 3, 2025 (KR) 10-2025-0089233

(51) Int. Cl. G16H 20/60; G16H 10/20; G16H 10/60; G16H 20/30; G16H 50/70

(52) U.S. Cl. CPC G16H 20/60; G16H 10/20; G16H 10/60; G16H 20/30; G16H 50/70

(57) ABSTRACT

The present disclosure relates to a method and apparatus for platform-based growth and dietary management support services for children and adolescents, and in particular, the method may include providing a target object with a questionnaire including a plurality of questions each related to body, diet, and exercise; obtaining answer information based on the questionnaire from the target object; inputting the answer information into a pre-trained artificial intelligence model to receive as an output goal information based on a score calculated according to a Mediterranean diet; setting at least one mission for the target object on the platform using the goal information; and monitoring whether to perform the at least one mission based on the input information when the input information for the target object is received.

### CROSS-REFERENCE TO RELATED APPLICATIONS

This application claims the priorities of Korean Patent Application No. 10-2024-0202950 filed on Dec. 31, 2024, and Korean Patent Application No. 10-2025-0089233 filed on Jul. 3, 2025, in the Ministry of Intellectual Property of the Republic of Korea, the disclosures of which are incorporated herein by reference.

### BACKGROUND

### Field

The present disclosure relates to a method and apparatus for platform-based growth and dietary management support services for children and adolescents.

### Description of the Related Art

With advances in medical technology, people's average life expectancy and life expectancy at birth are increasing, and interest in their health status and their health care is also increasing.

In addition, technologies for personal devices such as communication technology, sensor technology, and medical technology are also rapidly developing, and health care-related platforms and products based on user terminals are being released, and the demand for them is also rapidly increasing.

Moreover, since children and adolescents have different characteristics from adults, it is necessary to develop a technology that enables health care to be carried out based on a platform or product suitable for children and adolescents.

The background technology of the present invention was written to facilitate understanding of the present invention. It should not be understood that the matters described in the background of the invention exist as prior art.

### SUMMARY

Due to advances in smart health care technology and increasing desire for health, the recent health care industry is expanding its scope of application not only to disease treatment but also to health care and disease prediction. Individuals have become proactive in daily health care and health management, effectively managing obtained health information, and are increasingly interested in customized precision medicine and health care services. In particular, the era of experiencing the use of wearable IoT devices, disease prevention through genetic analysis and artificial intelligence (hereinafter, AI), and health care coaching (consulting) services has arrived. A considerable amount of big data regarding health care/health management is being generated, and technology development to analyze big data and creative business models using it are leading new innovations.

Accordingly, technologies for health-related management platforms that automatically or manually record a user's nutritional status based on smart devices such as smartphones or wearable devices and allow the user to visually check the analysis results are also being actively developed.

However, most of the platforms related to health care currently provided are platforms for adults, and advances in platforms suitable for children's developmental characteristics is sluggish.

In the case of children and adolescents, because the diseases and symptoms that may occur by age are different, it is essential to continuously manage the characteristics of the child or adolescent and provide an appropriate treatment and care environment, but the problem with existing platforms is that they only allow for one-time management, and continuous management according to changes in developmental and nutritional status is difficult.

Accordingly, the inventors of the present disclosure have recognized that when nutrition management for children and adolescents with chronic diseases is incorporated into smart health care technology, anyone can more easily prevent and manage children and adolescents with chronic diseases, and accordingly, there is a need for a method and apparatus for constructing a pipeline that provides a guidance for normal growth and development according to the characteristics of children and adolescents.

Accordingly, an object to be achieved by the present disclosure is to provide a method and apparatus for platform-based growth and dietary management support services for children and adolescents that enables normal growth by accurately grasping the developmental status and nutritional status through a questionnaire on each of body, diet, and exercise of children and adolescents, and by determining a target value according to the grasped developmental status and nutritional status and promoting achievement, thereby preventing the occurrence of diseases that occur frequently in children and adolescents.

On the other hand, another object to be achieved by the present disclosure is to provide a method and apparatus for platform-based growth and dietary management support services for children and adolescents that provides a mission to children and adolescents and, when it is achieved, rewards such as scores, and induces interests in children and adolescents through visual and auditory effects, thereby motivating them to perform the mission and enabling voluntary participation in health management activities.

Objects of the present disclosure are not limited to the above-mentioned objects, and other objects not mentioned will be clearly understood by those skilled in the art from the description below.

In order to solve the above-described problems, a method for platform-based growth and dietary management support services for children and adolescents according to an embodiment of the present disclosure is provided. The method may include providing a questionnaire including a plurality of questions each related to body, diet, and exercise to a target object; obtaining answer information based on the questionnaire from the target object; receiving goal information based on the developmental status and nutritional status of the target object as output based on the score calculated according to the Mediterranean diet by inputting the answer information into a pre-trained artificial intelligence model; setting at least one mission for the target object on the platform using the goal information; and monitoring, when input information for the target object is received, whether to perform the at least one mission based on the input information.

According to a feature of the present disclosure, the answer information may be obtained based on a questionnaire mapped to the age of the target object and body data measured by a pre-connected measuring terminal, the questionnaire may be divided into physical items, diet items, and exercise items, and is configured to include at least one questionnaire in each item, and the body data may include a measurement for at least one of weight, height, blood sugar, blood pressure, pulse, body fat, oxygen saturation, body temperature, heart rate, respiration rate, and electrocardiogram.

According to a feature of the present disclosure, the artificial intelligence model may calculate a KIDMED score using the answer information based on the Mediterranean diet and generate the goal information including a target weight value, a target performance value, a target intake amount, and at least one educational material using the calculated score.

According to a feature of the present disclosure, the artificial intelligence model may, when generating the goal information, determine the target weight value based on at least one of the body mass index and risk factors of the target object, identify recommended exercise items corresponding to the type and number of exercises performed by the target object, determine the target performance value for each exercise included in the recommended exercise items by considering the physical information of the target object, determine at least one nutrient that needs to be consumed and the intake ratio of each nutrient by identifying the intake problem of the target object, and determine the target intake amount for at least one of the foods and supplements containing each nutrient.

According to a feature of the present disclosure, the at least one mission may include at least one of whether training has been completed based on the at least one educational material, whether the target performance value for each exercise has been achieved, and whether the target intake amount for at least one of food and supplements containing each nutrient has been achieved.

According to a feature of the present disclosure, when determining the target weight value, the artificial intelligence model may further determine and provide any one of the multiple nutritional intervention methods by considering at least one of the body mass index and risk factors so as to reach the target weight value.

According to a feature of the present disclosure, the multiple nutritional intervention methods may include a method of setting an intake ratio of three major nutrients and a method of setting an intake ratio of all nutrients.

According to a feature of the present disclosure, the KIDMED score may be determined by automatically adding or subtracting a preset score for a positive or negative answer for each question in the questionnaire.

According to a feature of the present disclosure, the method may further include adding or subtracting the KIDMED score of the target object based on whether the at least one mission is performed, according to preset score information.

According to a feature of the present disclosure, the method may further include generating and providing a notification including an encouraging message or a compliment message in the form of at least one of a pop-up window, a voice message, and a text, based on a result of mission performance.

In order to solve the above-described problems, an apparatus for platform-based growth and dietary management support services for children and adolescents according to an embodiment of the present disclosure is provided. The apparatus comprises a communication interface; a memory; and a processor operably connected with the communication interface and the memory, the processor being configured to provide a questionnaire including a plurality of questions each related to body, diet, and exercise to a target object; obtain answer information based on the questionnaire from the target object; receive goal information based on the developmental status and nutritional status of the target object as output based on the score calculated according to the Mediterranean diet by inputting the answer information into a pre-trained artificial intelligence model; set at least one mission for the target object on the platform using the goal information; and monitor, when input information for the target object is received, whether to perform the at least one mission based on the input information.

According to a feature of the present disclosure, the answer information is obtained based on a questionnaire mapped to the age of the target object and body data measured by a pre-connected measuring terminal, the questionnaire is divided into physical items, diet items, and exercise items, and is configured to include at least one questionnaire in each item, and the body data may include a measurement for at least one of weight, height, blood sugar, blood pressure, pulse, body fat, oxygen saturation, body temperature, heart rate, respiration rate, and electrocardiogram.

According to a feature of the present disclosure, the artificial intelligence model may calculate a KIDMED score using the answer information based on the Mediterranean diet and generate the goal information including a target weight value, a target performance value, a target intake amount, and at least one educational material using the calculated score.

According to a feature of the present disclosure, the processor may, when generating the goal information, determine the target weight value based on at least one of the body mass index and risk factors of the target object, identify recommended exercise items corresponding to the type and number of exercises performed by the target object, determine the target performance value for each exercise included in the recommended exercise items by considering the physical information of the target object, determine at least one nutrient that needs to be consumed and the intake ratio of each nutrient by identifying the intake problem of the target object, and determine the target intake amount for at least one of the foods and supplements containing each nutrient.

According to a feature of the present disclosure, the at least one mission may include at least one of whether training has been completed based on the at least one educational material, whether the target performance value for each exercise has been achieved, and whether the target intake amount for at least one of food and supplements containing each nutrient has been achieved. According to a feature of the present disclosure, when determining whether to perform the at least one mission, the processor may be configured to determine whether the target performance value has been achieved by detecting the type and number of exercises performed by the target object when the input information is a video or image including the target object performing an exercise.

According to a feature of the present disclosure, when determining whether to perform the at least one mission, the processor may be configured to determine whether the target object has achieved the target intake amount by detecting the type and intake amount of food consumed, when the input information is a video or image including tableware before and after consumption by the target object.

According to a feature of the present disclosure, when determining the target weight value, the processor may be further configured to determine and provide any one of the multiple nutritional intervention methods by considering at least one of the body mass index and risk factors so as to reach the target weight value.

According to a feature of the present disclosure, the multiple nutritional intervention methods may include a method of setting an intake ratio of three major nutrients and a method of setting an intake ratio of all nutrients.

According to a feature of the present disclosure, the KIDMED score may be determined by automatically adding or subtracting a preset score for a positive or negative answer for each question in the questionnaire.

According to a feature of the present disclosure, the processor may be configured to add or subtract the KIDMED score of the target object based on preset score information, depending on whether the at least one mission is performed.

According to a feature of the present disclosure, the processor may be configured to generate and provide a notification including an encouraging message or a compliment message in the form of at least one of a pop-up window, a voice message, and text, according to a result of monitoring whether to perform the at least one mission.

Other detailed matters of the embodiments are included in the detailed description and the drawings.

The present disclosure may accurately identify the developmental status and nutritional status of a child through a questionnaire on each of the child's body, diet, and exercise, determine a target value according to the identified developmental status and nutritional status and promote achievement thereof, thereby preventing the occurrence of diseases that occur frequently in the child, thereby enabling normal growth.

In addition, the present disclosure provides a mission to a child, which, when achieved, provides rewards such as scores, and induces interest in the child through visual and auditory effects, thereby motivating the child to perform the mission and enabling voluntary participation in health care activities.

The effects according to the present disclosure are not limited to the contents exemplified above, and more various effects are included in the present disclosure.

### BRIEF DESCRIPTION OF DRAWINGS

FIG. 1 is a schematic diagram illustrating a system for platform-based growth and

dietary management support services for children and adolescents according to an embodiment of the present invention.

FIG. 2 is a block diagram illustrating a configuration of a service server that provides platform-based growth and dietary management support services for children and adolescents according to an embodiment of the present invention.

FIG. 3 is a block diagram illustrating a configuration of a user terminal for using platform-based growth and dietary management support services for children and adolescents according to an embodiment of the present invention.

FIG. 4 is a flowchart schematically illustrating a method for platform-based growth and dietary management support services for children and adolescents according to an embodiment of the present invention.

FIGS. 5A and 5B are views illustrating an example of a user interface implemented to input an answer to a questionnaire related to a diet of a target object through a user terminal according to an embodiment of the present invention.

FIGS. 5C and 5D are diagrams respectively illustrating an example of a questionnaire prepared by a medical staff and answer information for the questionnaire according to an embodiment of the present invention.

FIGS. 6A and 6B are diagrams illustrating an example of a user interface implemented to input answers to questionnaires related to body, diet, and exercise of a target object through a user terminal according to an embodiment of the present invention.

FIGS. 7A to 7C are diagrams illustrating an example of a user interface through which a medical staff may input body data of a target object through a medical staff terminal according to an embodiment of the present invention.

FIGS. 8A and 8B are views illustrating an example of a user interface through which a target object, a guardian, and/or a medical staff may receive a nutrition intake analysis result based on an answer to a questionnaire related to a diet of the target object through each terminal according to an embodiment of the present invention.

FIG. 9 is a diagram illustrating an example of an algorithm for determining a target weight value based on a body mass index and risk factors of a target object according to an embodiment of the present invention.

FIG. 10A is a diagram illustrating an example of an algorithm for determining a target performance value for each exercise by identifying recommended exercise items corresponding to the type and number of exercises performed by the target object according to a first embodiment of the present invention.

FIG. 10B is a diagram illustrating an example of an algorithm for determining a target performance value for each exercise by identifying recommended exercise items corresponding to the type and number of exercises performed by the target object according to a second embodiment of the present invention.

FIGS. 11A to 11C are diagrams illustrating an example in which at least one mission to be performed on a target object is set on a platform using goal information about the target object according to an embodiment of the present invention.

FIGS. 12A to 12C are diagrams illustrating an example in which a target object is provided by playing educational materials in a video form as one of at least one mission through a user terminal according to an embodiment of the present invention.

FIG. 13 is a diagram illustrating an example of an algorithm for recalculating and updating a KIDMED score according to whether a target object has reached a developmental state and/or a nutritional state according to an embodiment of the present invention.

FIGS. 14A and 14B are diagrams illustrating an example of a user interface through which a preset guardian of a target object may receive analysis information of the target object through a guardian terminal according to an embodiment of the present invention.

### DETAILED DESCRIPTION OF THE EMBODIMENT

The advantages and features of the present invention, and the methods for achieving them, will become clear with reference to the embodiments described in detail below together with the attached drawings. However, the present invention is not limited to the embodiments disclosed below, but may be implemented in various different forms, and these embodiments are provided only to make the disclosure of the present invention complete and to fully inform a person having ordinary knowledge in the technical field to which the present invention belongs of the scope of the disclosure, and the present invention is defined only by the scope of the claims. In relation to the description of the drawings, similar reference numerals may be used for similar components.

In the present disclosure, the expressions “have”, “can have”, “include”, or “can include” refer to the presence of a feature (for example, a numerical value, function, operation, or component such as a part) and do not exclude the presence of additional features.

In the present disclosure, the expressions “A or B”, “at least one of A or/and B”, or “one or more of A or/and B” may include all possible combinations of the items listed together. For example, “A or B”, “at least one of A and B”, or “at least one of A or B” may all refer to (1) including at least one A, (2) including at least one B, or (3) including both at least one A and at least one B.

The expressions “first”, “second”, “firstly”, or “secondly”, used in the present disclosure may describe various components, regardless of order and/or importance, and are used only to distinguish one component from another, but do not limit the components. For example, a first user device and a second user device may represent different user devices, regardless of order or importance. For example, without departing from the scope of the rights set forth in the present invention, a first component may be referred to as a second component, and similarly, a second component may be referred to as a first component.

When a component (for example, a first component) is referred to as being “(operatively or communicatively) coupled with/to” or “connected to” another component (for example, a second component), it should be understood that said component may be directly connected to said other component, or may be connected via another component (for example, a third component). Meanwhile, when it is said that a component (for example, a first component) is “directly connected” or “directly coupled” to another component (for example, a second component), it can be understood that no other component (for example, a third component) exists between said component and said other component.

The expression “configured to” as used in the present invention can be used interchangeably with, for example, “suitable for”, “having the capacity to”, “designed to”, “adapted to”, “made to”, or “capable of, depending on the situation. “The term “configured (or set) to” does not necessarily mean that something is “specifically designed to” in terms of hardware. Instead, in some contexts, the expression “an apparatus configured to” can mean that the apparatus is “capable of” doing something in conjunction with other apparatus or components. For example, the phrase “a processor configured (or set) to perform A, B, and C” may mean a dedicated processor (for example, an embedded processor) for performing the operations, or a general-purpose processor (for example, a CPU or an application processor) that can perform the operations by executing one or more software programs stored in a memory device.

The terms used in the present invention are used only to describe specific embodiments and may not be intended to limit the scope of other embodiments. The singular expression may include the plural expression unless the context clearly indicates otherwise. Terms used herein, including technical or scientific terms, may have the same meaning as commonly understood by a person of ordinary skill in the art described in the present invention. Terms defined in general dictionaries among the terms used in the present invention may be interpreted as having the same or similar meaning as they have in the context of the relevant technology, and shall not be interpreted in an ideal or excessively formal sense unless explicitly defined in the present invention. In some cases, even if a term is defined in the present invention, it cannot be interpreted to exclude embodiments of the present invention.

Each feature of the various embodiments of the present invention can be partially or entirely combined or combined with each other, and as can be fully understood by those skilled in the art, various technical connections and operations are possible, and each embodiment can be implemented independently of each other or can be implemented together in a related relationship.

For clarity in the interpretation of this specification, the terms used in this specification are defined below.

A device referred to as a “service server” below may refer to one physically independent server according to the present disclosure, but is not limited thereto, and may be a single virtual machine, and is intended to cover all one module, program, or docker operating in one virtual or physical machine.

Hereinafter, preferred embodiments of the present disclosure will be described in detail with reference to the accompanying drawings.

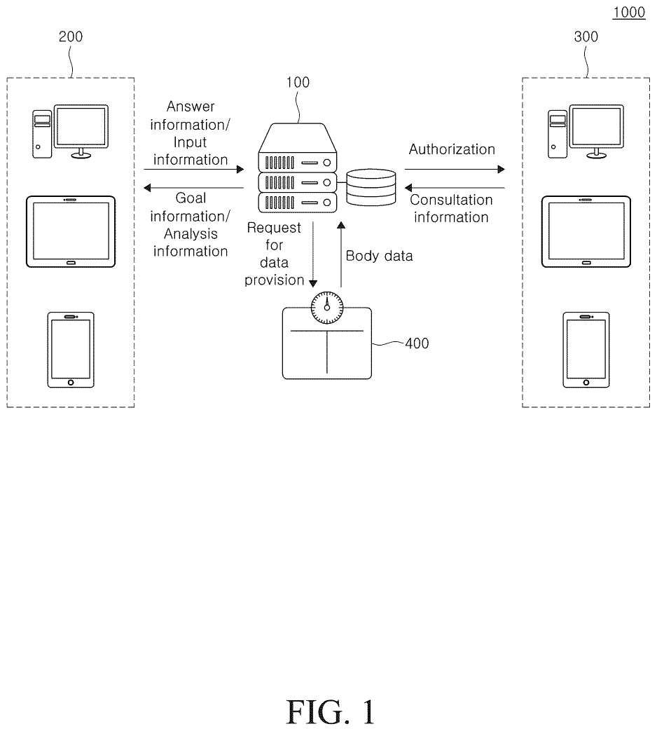

FIG. 1 is a schematic diagram illustrating a system for platform-based growth and dietary management support services for children and adolescents according to an embodiment of the present invention.

Referring to FIG. 1, a system 1000 for providing platform-based growth and dietary management support services for children and adolescents according to an embodiment of the present invention (hereinafter, referred to as a “service providing system”) may be a system configured to analyze a developmental state and a nutritional state of the child or adolescent object (hereinafter, referred to as a “target object”) to provide a suitable mission and induce the target object to actively perform the mission to prevent diseases that may occur in the target object. Accordingly, the service providing system 1000 may include a service server 100, a user terminal 200, a medical staff terminal 300, and a measurement terminal 400.

First, the service server 100 may correspond to a web server, and refers to an apparatus for providing growth and dietary management support services (hereinafter, referred to as a ‘management support service’) for children and adolescents (i.e., an apparatus for supporting growth and dietary management for children and adolescents) in the service providing system 1000 of the present invention.

In order to provide the management support service, the service server 100 may be connected to the user terminal 200, the medical staff terminal 300, the measurement terminal 400, etc. and collect or receive all information/data related to the management support service such as various notifications, requests, and information. Meanwhile, the service providing system may be linked (connected) with the electronic medical record system in order to collect and use various information about each target object. This electronic medical record system is configured to allow each medical staff or each medical institution to record and manage various information about each target object, and when information about a specific target object is input from a specific medical staff or a specific medical institution, all of them can be processed and managed as a database. Here, various types of information about each target object may include basic information such as personal information and physical information, as well as medical information including at least one of medical history, treatment results, and medical data. However, this is merely an embodiment, and may further include other additional information about the target object, or at least some information may be excluded, and the stored data/information is not limited to the previously listed data/information.

Specifically, the service server 100 obtains answer information by providing the user terminal 200 with a questionnaire corresponding to the age of the target object among the pre-stored questionnaires related to each body, diet, and exercise, and sets at least one mission on the platform by using the goal information based on the developmental status and nutritional status of the target object generated as a result of the analysis of the answer information. This allows the target object to perform at least one mission based on the platform. Here, the goal information is at least one target value that the target object must achieve in relation to body, diet, and exercise, and may include at least one of a target weight value, a target performance value for each exercise, and a target intake amount for food/supplement containing each nutrient. In addition, the goal information may further include at least one educational material related to an exercise and a diet to be referenced by the target object, wherein each educational material may be in the form of at least one of a video, a voice, a document, an image, or a link.

To this end, the service server 100 may include at least one artificial intelligence model pre-trained to analyze the answer information. Accordingly, the service server 100 may input the answer information received from the user terminal 200 to the artificial intelligence model to receive the goal information based on the developmental state and nutritional state of the target object as an output.

Further, in addition to the answer information based on the questionnaire, the service server 100 may receive information or data input through the medical staff terminal 300 as the consultation information a consultation on the target object performed by a medical staff to which treatment authority or management authority is granted for the target object.

In addition, the service server 100 may request the pre-linked (connected) measurement terminal 400 to provide data, thereby obtaining body data measured for the target object. In this case, although the measurement terminal 400 is separately illustrated in FIG. 1, it may be included in the user terminal 200 and/or the medical staff terminal 300. That is, the body data for the target object may be measured by at least one of the user terminal 200 and the medical staff terminal 300, and the measured body data may be transmitted to the service server 100.

In other words, the service server 100 may obtain various types of information related to each of body, diet, and exercise for the target object based on at least one of answer information and consultation information, and further obtain body data measured by the measurement terminal 400. In this case, the collected various pieces of information and data (including body data) may be entered using a pre-stored template and stored and managed in a database.

On the other hand, when input information is received from the user terminal 200, the service server 100 determines whether to perform each of at least one mission for the target object set on the platform based on the input information. Further, the service server 100 may not only output and provide notifications in at least one way based on the determination result, but may also motivate the target object to perform the mission through a reward procedure such as calculating scores based on a preset algorithm (method) or providing rewards.

Furthermore, although not shown in FIG. 1, the service server 100 may register at least one guardian terminal 500 of the guardian(s) corresponding to the target object in advance and transmit and provide analysis information about the target object to each guardian terminal 500. Here, the analysis information may include an analysis result for each of the developmental state and nutritional state of the target object.

This guardian terminal 500 may be a concept included in the user terminal 200, but it is described using separate terms to distinguish it from the user terminal so that the target object can perform each mission.

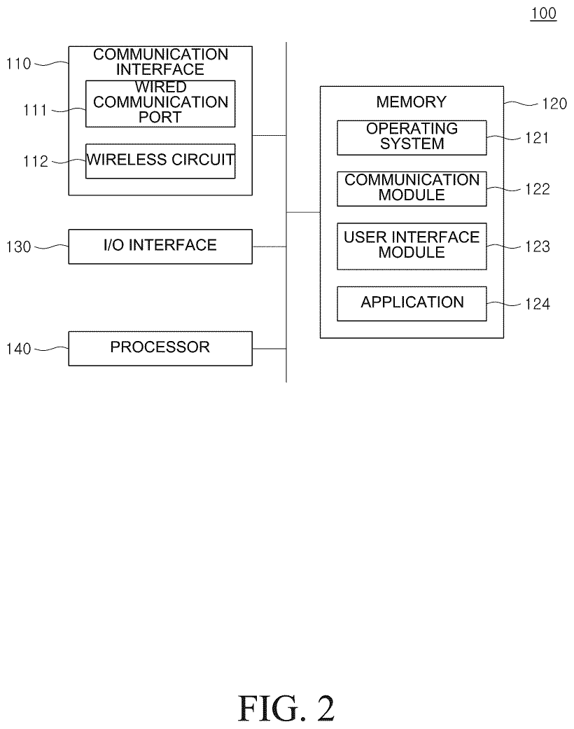

This service server 100 will be described in detail later with reference to FIG. 2.

Meanwhile, both the user terminal 200 and the medical staff terminal 300 are terminals possessed by each user or each medical staff registered in advance on the service server 100 to be provided with (to use) management support services. In this case, each of the user terminal 200 and the medical staff terminal 300 may be provided with a management support service by executing a separate platform (application) provided by the service server 100.

First, the user terminal 200 collectively refers to a terminal possessed by a target object or a guardian who wants to be provided with a management support service, and may be at least one or more. The target object may execute the platform through the user terminal 200 to receive a questionnaire for each of body, diet, and exercise, enter an answer based on the questionnaire, and transmit the answer information generated as a result to the service server 100. In addition, the target object may receive goal information and/or analysis information generated based on the answer information through the user terminal 200, and when input information for each of at least one mission set based on them is input, the target object may transmit the input information to the service server 100. In this case, the input information may be in the form of at least one of text, numbers, emoticons, audio, images, and videos, and may be automatically or manually recorded according to a template provided through the platform.

Specifically, when the target object captures and uploads at least one of a video/image showing the target object measuring their own body data, a video/image showing the target object performing exercise, and a video/image showing the object's tableware before and after food consumption as input data to the platform via the user terminal 200, the service server 100 can analyze it and automatically record the analysis results. Accordingly, the service server 100 may determine whether the target object has achieved the target weight value, the target performance value, and/or the target intake amount based on the analysis result.

To this end, the user terminal 200 may include at least one capturing device and at least one sensor. Here, the at least one capturing device may include at least one of a 2D camera, a 3D camera, a Time of Flight (ToF) camera, a light field camera, a stereo camera, an event camera, an infrared camera, a lidar sensor, and an array camera, through which depth information may be measured based on an image or an image to predict a weight thereof. In other words, the service server 100 may automatically recognize (detect) and record at least one of name, weight, material, calories, and nutrients as data for each food consumed by the target object. In addition, when the service server 100 receives a voice input for each food consumed by the target object, the voice may be recognized through an artificial intelligence model and automatically recorded. In addition, the target object may directly manually input and record data for each of the at least one food. That is, because of the characteristics of children and adolescents, it may be difficult to manually input a record of the food ingested by the child or adolescent, so they upload the video or image or speak, the service server 100 may analyze that to automatically input a record of the food ingested by the target object.

In addition, the target object may provide the biometric data collected through at least one sensor provided in the user terminal 200 to the service server 100 so that the biometric data is automatically input. Here, the biometric data may include at least one of blood sugar, blood pressure, pulse, body fat, oxygen saturation, body temperature, heart rate, respiratory rate, and electrocardiogram.

Meanwhile, the medical staff terminal 300 collectively refers to a terminal possessed by a medical staff who consults with a target object face-to-face or remote, and may be at least one or more. At this time, the consultation may be performed to obtain various types of information that are indicators that can confirm the developmental status and nutritional status of the target object, or may be performed in parallel based on treatment due to a disease.

That is, the medical staff may run the platform through the medical staff terminal 300 to directly survey each of body, diet, and exercise, enter the answer of the target object or guardian to the questionnaire, and transmit the generated consultation information to the service server 100.

Each of the aforementioned user terminal 200 and/or medical staff terminal 300 may be one or more devices. Each device may be a computer, UMPC (Ultra Mobile PC), workstation, net-book, Personal Digital Assistants (PDAs), portable computer, web tablet, wireless phone, mobile phone, smart phone, pad, smart watch, wearable terminal, e-book reader, portable multimedia player, portable game console, navigation device, black box, digital camera, or other mobile communication terminal capable of installing and executing a plurality of applications desired by each target object (or guardian) and/or medical staff, without being limited thereto.

Meanwhile, the measurement terminal 400 may be a terminal or device pre-connected (linked) to the service server 100 to provide a management support service, and may be provided in a specific space such as a medical institution, or may be provided in at least one of the user terminal 200 and the medical staff terminal 300.

This measurement terminal 400 may include at least one of weight, height, body fat mass, body mass index, muscle mass, muscle mass index, and phase angle as body data for the target object, as well as at least one of blood sugar, blood pressure, pulse, body fat, oxygen saturation, body temperature, heart rate, respiratory rate, and electrocardiogram. To this end, the measurement terminal 400 may include at least one sensor for measuring (collecting) body data.

Meanwhile, the service providing system 1000 is not limited to the configuration illustrated in FIG. 1, and may be configured to include further other devices (terminals, servers, etc.) or may be configured except for some configurations.

Based on such a service providing system 1000, it is possible to accurately grasp the developmental status and nutritional status through a questionnaire on each of body, diet, and exercise of children and adolescents, determine a target value based on the grasped developmental status and nutritional status, and promote achievement, thereby preventing the occurrence of diseases that frequently occur in children and adolescents, thereby enabling normal growth.

FIG. 2 is a block diagram illustrating a configuration of a service server that provides platform-based growth and dietary management support services for children and adolescents according to an embodiment of the present invention.

Referring to FIG. 2, the service server 100 may include a communication interface 110, a memory 120, an I/O interface 130, and a processor 140, and each component may communicate with each other through one or more communication buses or signal lines.

The communication interface 110 may be connected to the user terminal 200, the medical staff terminal 300, and the measurement terminal 400, as well as other devices, to exchange data through a wired/wireless communication network.

Meanwhile, a communication interface 110 enabling transmission and reception of such data includes a wired communication port 111 and a wireless circuit 112, wherein the wired communication port 111 may include one or more wired interfaces, for example, Ethernet, a universal serial bus (USB), a Firewire, and the like. In addition, the wireless circuit 112 may transmit and receive data to and from an external device through an RF signal or an optical signal.

In addition, wireless communication may use at least one of multiple communication standards, protocols and technologies, such as GSM, EDGE, CDMA, TDMA, Bluetooth, Wi-Fi, VOIP, Wi-MAX, or any other suitable communication protocol.

The memory 120 may store data for at least one process (algorithm) for providing a management support service or a program reproducing the process. In addition, the memory 120 may further store processes for performing other operations, and is not limited thereto.

Meanwhile, the memory 120 may build and store a pre-trained model as needed as well as various data used in the service server 100.

In various embodiments, memory 120 may include a volatile or nonvolatile recording medium capable of storing various data, commands, and information. For example, the memory 120 may include at least one type of storage medium among flash memory type, hard disk type, multimedia card micro type, card type memory (for example, SD or XD memory, etc.), RAM, SRAM, ROM, EEPROM, PROM, network storage, cloud, and blockchain database.

In various embodiments, the memory 120 may store the configuration of at least one of the operating system 121, the communication module 122, the user interface module 123, and one or more applications 124.

Operating system 121 (e.g. embedded operating systems such as LINUX, UNIX, MAC OS, WINDOWS, VxWorks, etc.) may include various software components and drivers for controlling and managing general system tasks (e.g., memory management, storage device control, power management, etc.), and may support communication between various hardware, firmware, and software components.

The communication module 122 may support communication with other devices through the communication interface 110. The communication module 122 may include various software components for processing data received by the wired communication port 111 or the wireless circuit 112 of the communication interface 110.

The user interface module 123 may receive a viewer's request or input from a keyboard, a touch screen, a microphone, etc. through the I/O interface 130, and provide a user interface on the display.

Application 124 may include programs or modules configured to be executed by one or more processors 140.

The I/O interface 130 may connect at least one of input/output devices (not shown) of the service server 100, e.g., a display, a keyboard, a touch screen, and a microphone, to the user interface module 123. The I/O interface 130 may receive a user input (e.g., voice input, keyboard input, touch input, etc.) together with the user interface module 123 and process a command according to the received user input.

The processor 140 may be connected to the communication interface 110, the memory 120, and the I/O interface 130 to control the overall operation of the service server 100 and perform various commands through an application or a program stored in the memory 120.

The processor 140 may correspond to a computing device such as a central processing unit (CPU) or an application processor (AP). In addition, the processor 140 may be implemented in the form of an integrated chip (IC) such as a system on chip (SoC) in which various computing devices are integrated. Alternatively, the processor 140 may include a module for calculating an artificial neural network model, such as a neural processing unit (NPU).

Specifically, when the target object is registered on the platform, the processor 140 is configured to obtain answer information related to each of body, diet, and exercise of the target object from the user terminal 200, and to receive the goal information based on the developmental state and nutritional state of the target object as an output by inputting the answer information to a pre-trained artificial intelligence model. Here, the answer information may be obtained based on a questionnaire mapped (corresponding) to the age of the target object, which may be divided into body items, diet items, and exercise items and configured to include at least one questionnaire in each item. In this case, consultation information may be obtained from the medical staff terminal 300 instead of the answer information, or the answer information and the consultation information may be received from the user terminal 200 and the medical staff terminal 300, respectively, and used together. Further, each of the answer information and the consultation information may include body data measured by the measurement terminal 400 or may be obtained based on the body data. That is, each information about the target object for generating the goal information may be obtained in various ways, it is not limited thereto.

Thereafter, the processor 140 is configured to set at least one mission for the target object on the platform using the goal information, and when input information for the target object is received, determine whether to perform the at least one mission based on the input information, and output a notification according to the determination result.

To this end, the processor 140 may construct and include at least one pre-trained artificial intelligence model as described above.

For example, when generating the goal information, the artificial intelligence model may determine the target weight value based on the body mass index and risk factors of the target object, and after checking the recommended exercise item corresponding to the type and number of exercises performed by the target object, determine the target performance value for each exercise included in the recommended exercise item by considering the body information of the target object. Further, the artificial intelligence model can determine at least one nutrient which needs to be ingested and an intake ratio of each nutrient by identifying an intake problem of a target object, and determine a target intake amount for at least one of a food or supplements containing each nutrient. In other words, target values suitable for the developmental state and/or nutritional state are determined so as not to strain the target object.

In addition, at least one mission may be determined based on at least one of whether training has been completed based on at least one educational material, whether a target performance value for each exercise has been achieved, and whether a target intake amount for at least one of food and supplements containing each nutrient has been achieved.

Meanwhile, in determining whether to perform at least one mission, the processor 140 may determine whether the target weight value has been achieved, when a video or image including a target object measuring body data through a measurement terminal 400 is uploaded as input information based on the changed body data of the target object from the video or image.

Further, in determining whether to perform at least one mission, the processor 140 may determine whether the target performance value has been achieved by detecting the type and number of exercises performed by the target object, when an image or video including the target object performing an exercise is uploaded as input information, based on the image or video.

In addition, in determining whether to perform at least one mission, the processor 140 may determine whether a target intake amount has been achieved by detecting the type and intake amount of food ingested by the target object, when an image or video including tableware before and after the target object consumes food is uploaded as input information, based on the image or video.

In addition, when the target object completes training on at least one educational material, the processor 140 may calculate a score based on a pre-stored scoring condition for each training and add or subtract the score of the target object (or accumulated score) to the calculated score. Here, the at least one educational material may be stored in advance according to the age-specific developmental status and nutritional status of the target object, or provided as educational materials attached via the medical staff terminal 300 based on the analysis information of the target object by the medical staff.

Meanwhile, the processor 140 may receive and store the recommendation as a voice or text of the target object in order to remind the target object of the recommendation included in the goal information. For example, the processor 140 may have the target object to read or write words, sentences, etc. related to the recommendations, or may obtain a signature as a pledge thereto.

Further, when making a notification according to the determination result, the processor 140 outputs and provides an encouraging message or a compliment message in the form of at least one of a pop-up window, a voice message, and a text.

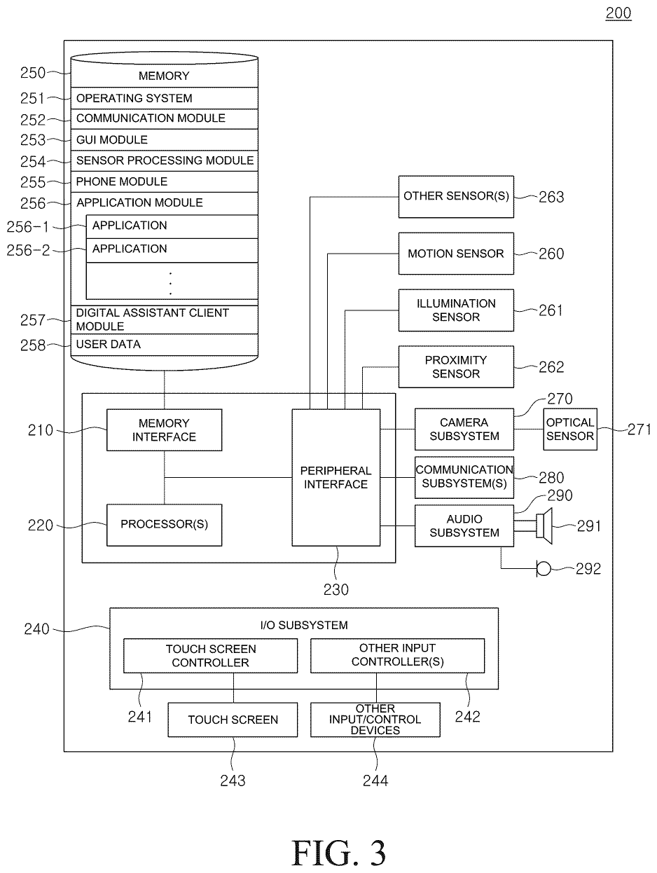

FIG. 3 is a block diagram illustrating a configuration of a user terminal for using platform-based growth and dietary management support services for children and adolescents according to an embodiment of the present invention. The configuration of the user terminal 200 may be equally applied to the medical staff terminal 300 and/or the measurement terminal 400, but will be described below only as the user terminal 200 for convenience of description.

Referring to FIG. 3, the user terminal 200 may include a memory interface 210, one or more processors 220, and a peripheral interface 230. Various components in the user terminal 200 may be connected by one or more communication buses or signal lines.

The memory interface 210 may be connected to the memory 250 to transmit various data to the processor 220. Here, the memory 250 may include at least one type of storage medium among flash memory type, hard disk type, multimedia card micro type, card type memory (for example, SD or XD memory, etc.), RAM, SRAM, ROM, EEPROM, PROM, network storage, cloud, and blockchain database.

In various embodiments, memory 250 may store a web/app application or program for providing a management support service. In addition, the memory 250 may store various types of information about the target object, and may store various types of information obtained through an application or a program.

In various embodiments, the memory 250 may store at least one of an operating system 251, a communication module 252, a graphical user interface module (GUI) 253, a sensor processing module 254, a telephone module 255, and an application module 256. Specifically, the operating system 251 may include instructions for processing a basic system service and instructions for performing hardware tasks. Communication module 252 may communicate with at least one of another one or more devices, computer, and server. The graphic user interface module GUI 253 may process a graphic user interface. The sensor processing module 254 may execute sensor-related functions (e.g., processing voice input received through one or more microphones 292). The telephone module 255 may execute telephone-related functions. The application module 256 may perform various functions of a user application, such as electronic messaging, web browsing, media processing, browsing, imaging, and other process functions. In addition, the user terminal 200 may store one or more software applications 256-1, 256-2 associated with any one type of service (e.g., a service application) in the memory 250.

In various embodiments, a memory 250 may store a digital assistant client module 257 (hereinafter, referred to as a DA client module), and accordingly, may store instructions and various user data 258 (e.g., user customized vocabulary data, preference data, and other data such as a user's electronic address book) for performing functions on the client side of the digital assistant.

Meanwhile, the DA client module 257 may obtain voice input, text input, touch input, and/or gesture input of the target object via various user interfaces (e.g., I/O subsystem 240) provided in the user terminal 200.

In addition, the DA client module 257 may output audio-visual and tactile types of data. For example, the DA client module 257 may output data including a combination of at least two of voice, sound, notification, text message, menu, graphic, video, animation, and vibration. In addition, the DA client module 257 may communicate with a digital assistant server (not shown) using the communication subsystem 280.

In various embodiments, the DA client module 257 can collect additional information about the surrounding environment of the user terminal 200 from various sensors, subsystems, and peripheral devices to configure a context associated with the user input. For example, the DA client module 257 can infer a user's intention by providing context information together with a user input to the digital assistant server. Here, the context information that can be accompanied by a user input can include sensor information, for example, lighting, ambient noise, ambient temperature, images of the surrounding environment, video, and the like. For another example, the context information can include a physical state (e.g., device orientation, device location, device temperature, power level, speed, acceleration, motion pattern, cellular signal strength, etc.) of the user terminal 200. As another example, the context information can include information related to the software status of the user terminal 200 (e.g., processes running on the user terminal 200, installed programs, past and current network activity, background services, error logs, resource usage, etc.).

In various embodiments, the memory 250 can include additional or deleted instruction. Further, the user terminal 200 can include additional configurations in addition to the configuration shown in FIG. 3, or can exclude some configurations.

The processor 220 can control the overall operation of the user terminal 200 and can execute various commands for using the management support service provided by the service server 100 by running an application or a program stored in the memory 250.

The processor 220 may correspond to a computing device such as a central processing unit (CPU) or an application processor (AP). In addition, the processor 220 may be implemented in the form of an integrated chip (IC) such as a System on Chip (SoC) in which various computing devices for performing machine learning, such as a Neural Processing Unit (NPU), are integrated.

In various embodiments, the processor 220 may receive various information and/or data on a target object based on a platform through a user interface screen.

The peripheral interface 230 may be connected to various sensors, subsystems, and peripheral devices to provide data so that the user terminal 200 may perform various functions. Here, it may be understood that a function performed by the user terminal 200 is performed by the processor 220.

The peripheral interface 230 can receive data from the motion sensor 260, the lighting sensor (light sensor) 261, and the proximity sensor 262 so that the user terminal 200 can perform orientation, light, and proximity sensing functions. For another example, the peripheral interface 230 can receive data from other sensors 263 (positioning system-GPS receiver, temperature sensor, biometric sensor), which can allow the user terminal 200 to perform functions related to other sensors 263.

In various embodiments, the user terminal 200 can include a camera subsystem 270 connected with the peripheral interface 230 and an optical sensor 271 connected thereto, such that the user terminal 200 can perform various photographing functions such as photographing and video clip recording.

In various embodiments, user terminal 200 can include communication subsystem 280 connected to peripheral interface 230. The communication subsystem 280 consists of one or more wired/wireless networks and can include various communication ports, radio frequency transceivers, and optical transceivers.

In various embodiments, the user terminal 200 includes an audio subsystem 290 associated with the peripheral interface 230, and such audio subsystem 290 includes one or more speakers 291 and one or more microphones 292, such that the user terminal 200 can perform voice-operated functions, such as voice recognition, voice reproduction, digital recording, and telephone functions.

In various embodiments, user terminal 200 can include I/O subsystem 240 connected to peripheral interface 230. For example, the I/O subsystem 240 can control the touch screen 243 included in the user device 200 through the touch screen controller 241.

For example, the touch screen controller 241 can detect a user's contact and movement or cessation of contact and movement by using any one of a plurality of touch sensing technologies such as capacitive, resistive, infrared, surface acoustic wave technology, proximity sensor array, etc. As another example, the I/O subsystem 240 can control other input/control devices 244 included in the user terminal 200 through other input controller(s) 242. As an example, the other input controller(s) 242 can control pointer devices such as one or more buttons, a rocker switch, a thumb-wheel, an infrared port, a USB port, and a stylus.

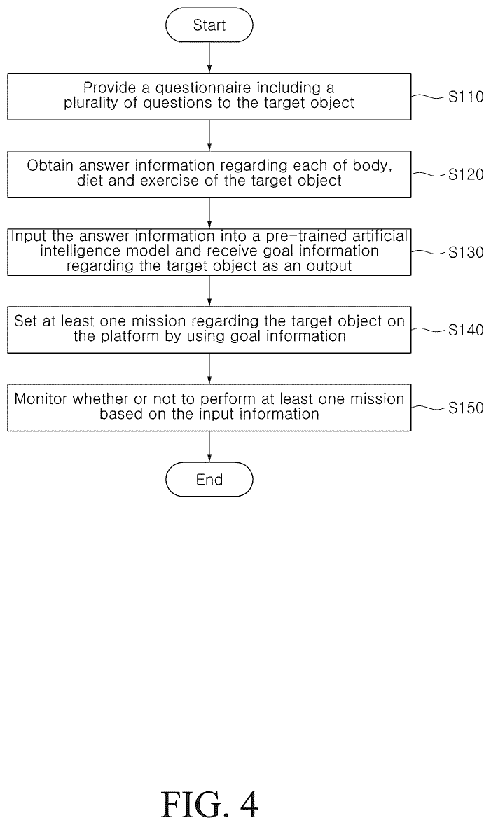

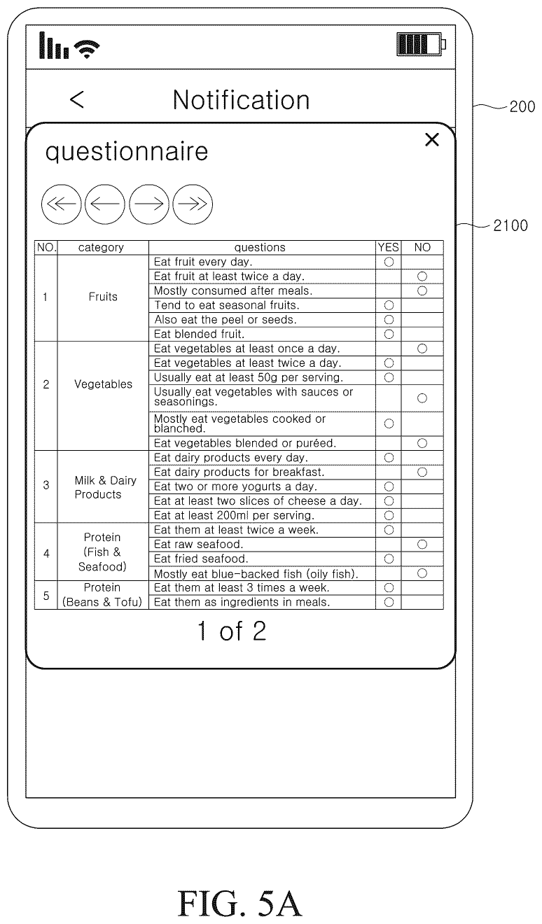

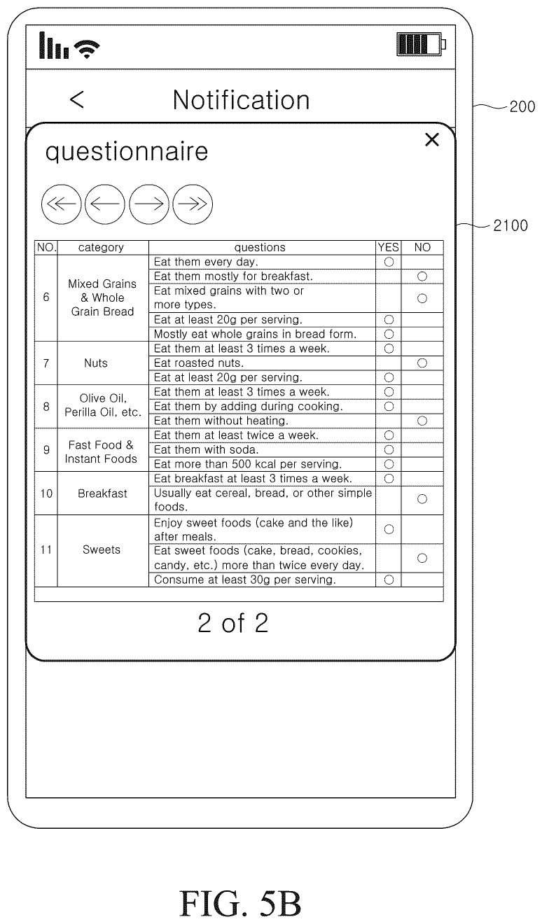

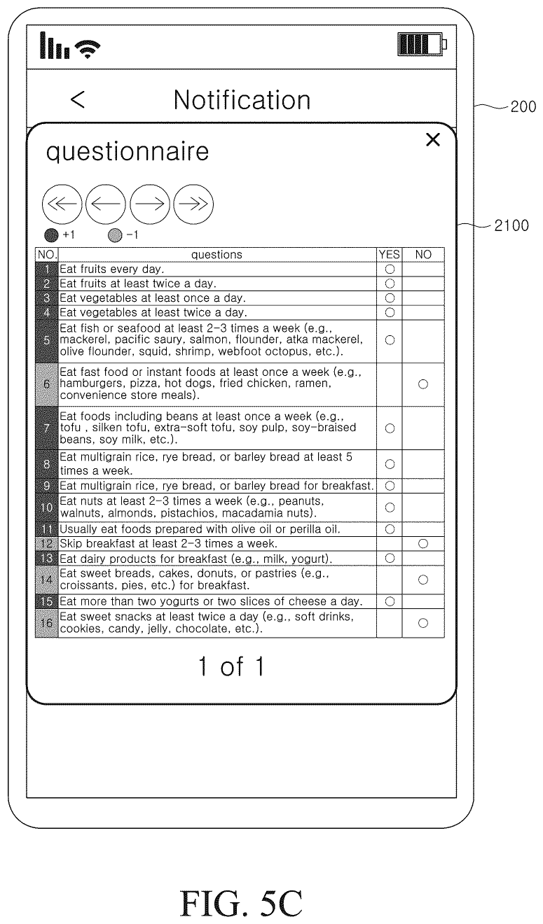

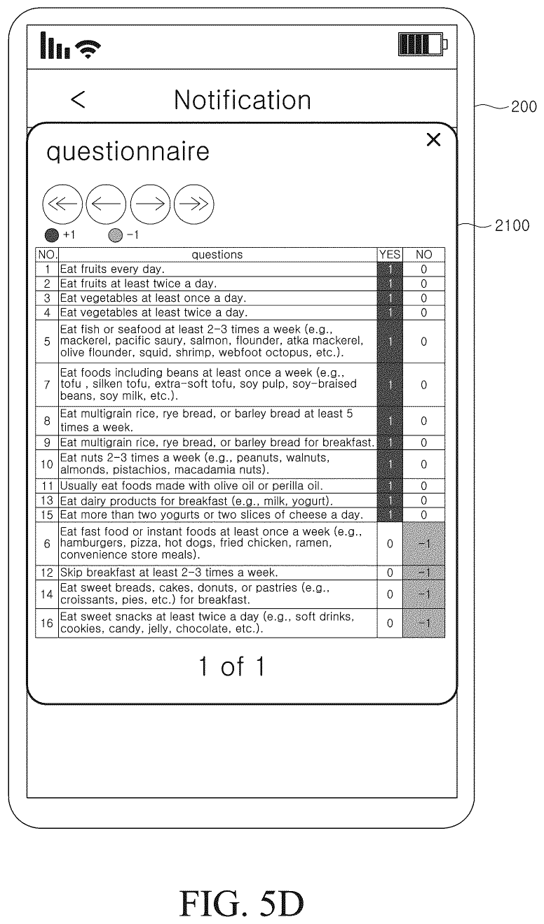

FIG. 4 is a flowchart schematically illustrating a method for platform-based growth and dietary management support services for children and adolescents according to an embodiment of the present invention, and a series of operations may be performed based on a platform built by the service server 100. Hereinafter, specific operations and contents will be described based on an example with reference to FIGS. 5A to 14C while explaining each step of FIG. 4.

Referring to FIG. 4, when a service provision request is received from a target object to be analyzed through a platform, the processor 140 provides a questionnaire including a plurality of questions related to each of body, diet, and exercise of the target object in order to register the target object (S110). As illustrated in FIGS. 5A and 5B, the target object may generate answer information by inputting or selecting an answer to each question based on the questionnaire displayed and provided through the display module 2100 of the user terminal 200. Here, a score may be given, or an encouraging message or a compliment message may be determined according to an answer to each question.

In this case, FIGS. 5A and 5B illustrate only questions about diet items, but in the questionnaire, questions about body items and/or exercise items may be further displayed and provided together.

The plurality of questions included in this questionnaire and the score for each answer to each questionnaire may be preset or may be set (added or edited) as necessary by the medical staff, and the setting method or score calculation method is not limited.

As an example, the medical staff may select only some of the questions from the pre-stored questionnaire as shown in FIG. 5A for specific children and adolescents and prepare a questionnaire as shown in FIG. 5C, and for each questionnaire, the score may be calculated by a score calculation method in which one point may be added by the positive answer, and one point may be deducted by the negative answer. Accordingly, answers to each questionnaire may be selected or input from the target object based on the questionnaire as shown in FIG. 5C to generate answer information as shown in FIG. 5D.

Such answer information may be sorted as it is according to the order of the questions, but may be sorted into positive and negative answers as shown in FIG. 5D.

Accordingly, when answer information corresponding to the questionnaire is received from a specific target object, a score may be calculated according to the score calculation method. If the answer information as shown in FIG. 5D is received, 12 points may be added by 12 positive answers and 4 points may be deducted by 4 negative answers. Finally, the KIDMED score of the specific target object may be determined to be 8 points.

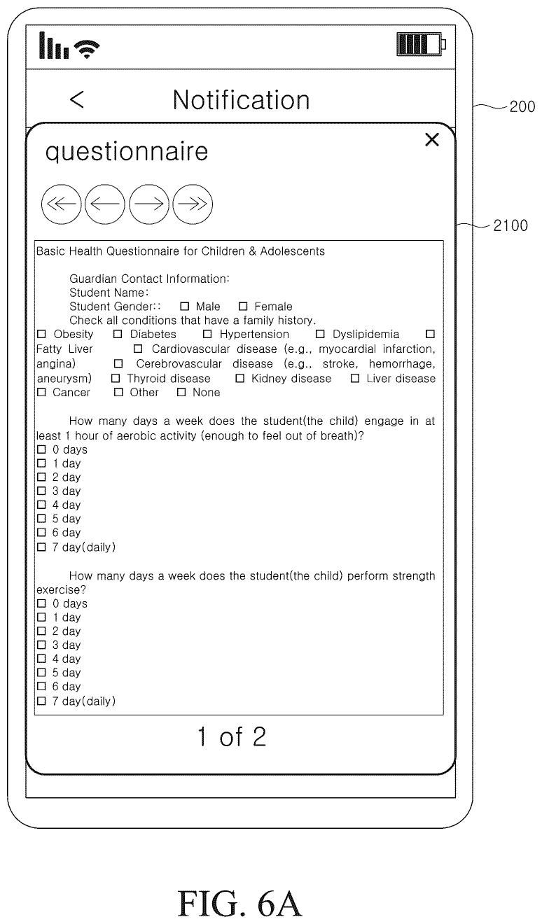

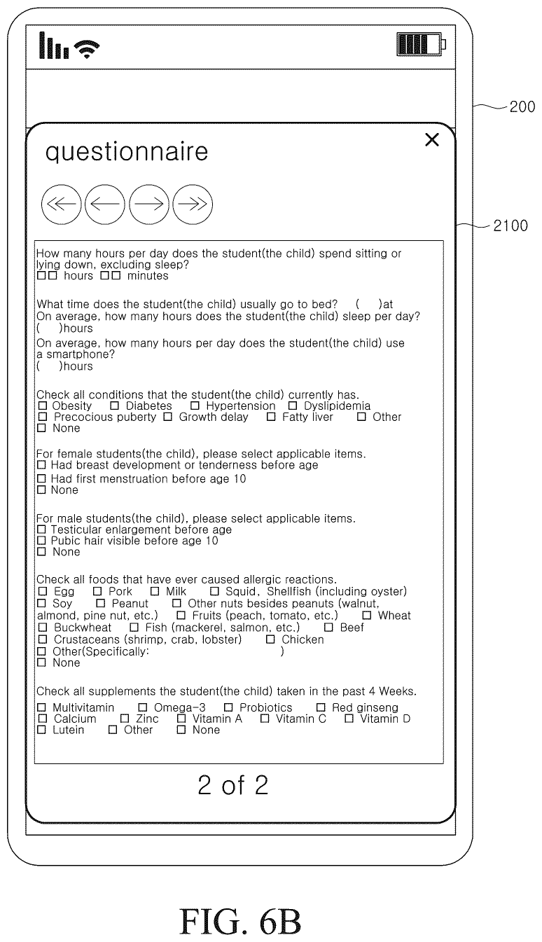

As another embodiment, as shown in FIGS. 6A and 6B, A guardian may generate answer information by entering or checking answers to each questionnaire based on a questionnaire for guardians displayed and provided through a display module 2100 of a user terminal 200 (or guardian terminal 500). That is, the questionnaire for a guardian may be configured to enter or check answers to questions related to body, diet, and exercise about the target object observed by the guardian.

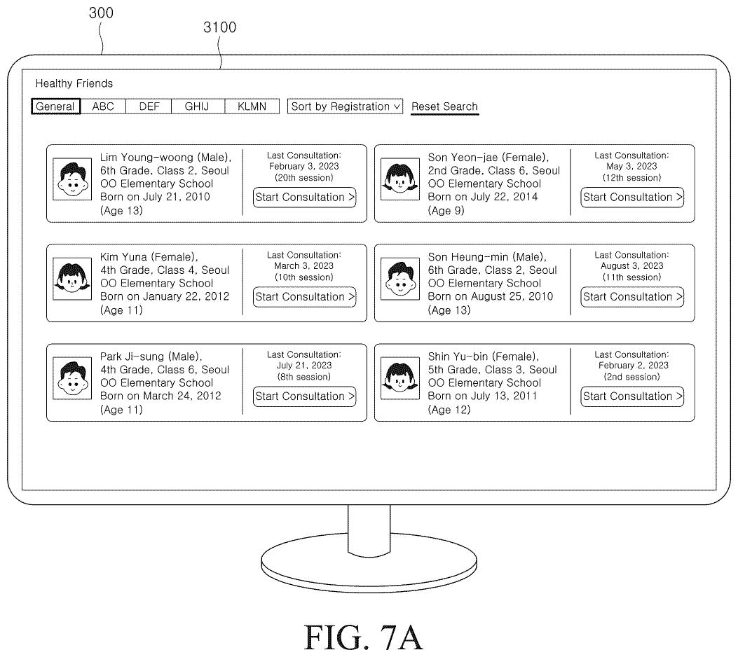

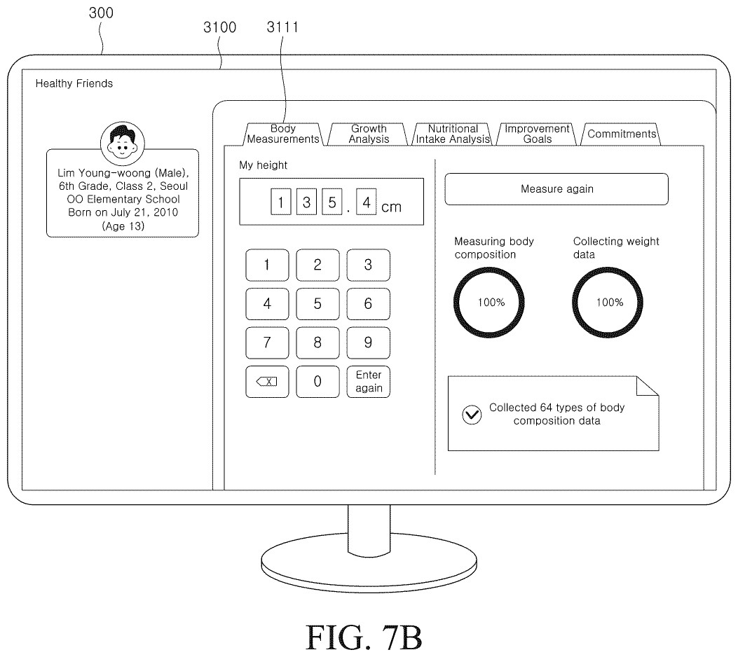

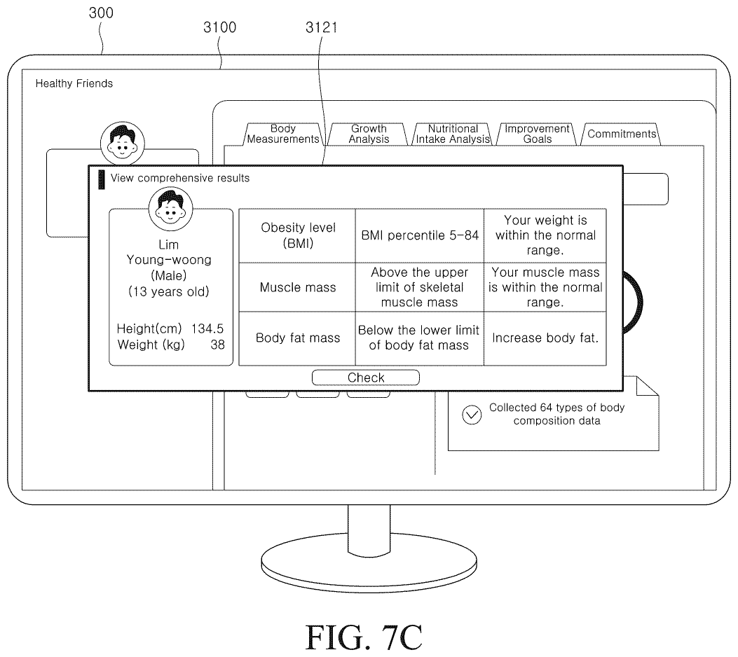

As another embodiment, as shown in FIGS. 7A to 7C, consultation information may be generated by a medical staff by entering or checking consultation contents based on a template displayed through the display module 3100 of the medical staff terminal 300. At this time, since the medical staff may manage a plurality of target objects, as shown in FIG. 7A, a template configured to enter or check information/data collected through the consultation may be retrieved by selecting a target object for consultation among the plurality of target objects. As shown in FIG. 7B, this template may be configured to allow medical staff enter or check answers to questions related to body, diet, and exercise about the target object based on answers obtained while conducting face-to-face or non-face-to-face consultations with the target object or guardian in the body measurement category 3111. In addition, as the consultation information is uploaded, a brief analysis result of the body data generated by the service server 100 may be provided through a separate pop-up window 3121 as shown in FIG. 7C. In this case, based on the pre-stored reference information, the analysis result may be obtained by comparing the levels of obesity (body mass index; BMI), muscle mass, and body fat mass to determine whether it is normal. However, this is merely an embodiment, and information included in the analysis result may be set or changed by a medical staff or a manager of the service server 100.

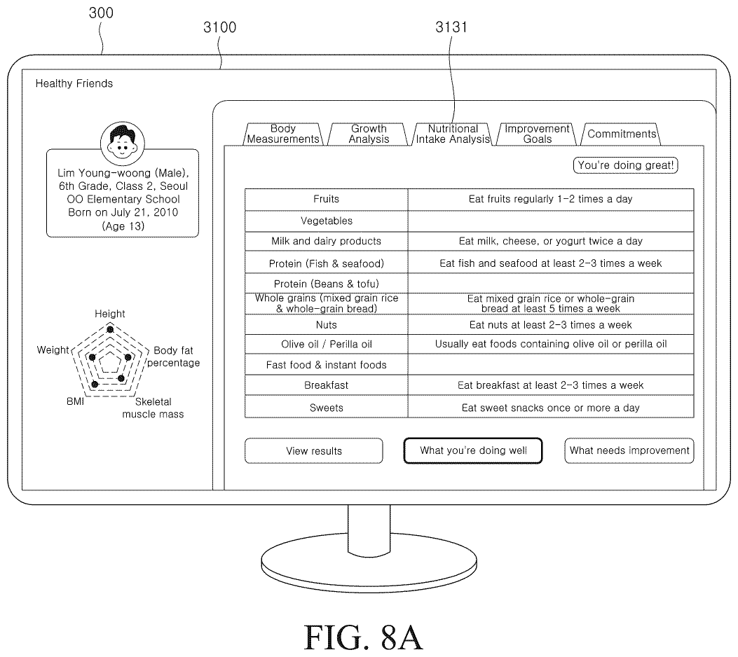

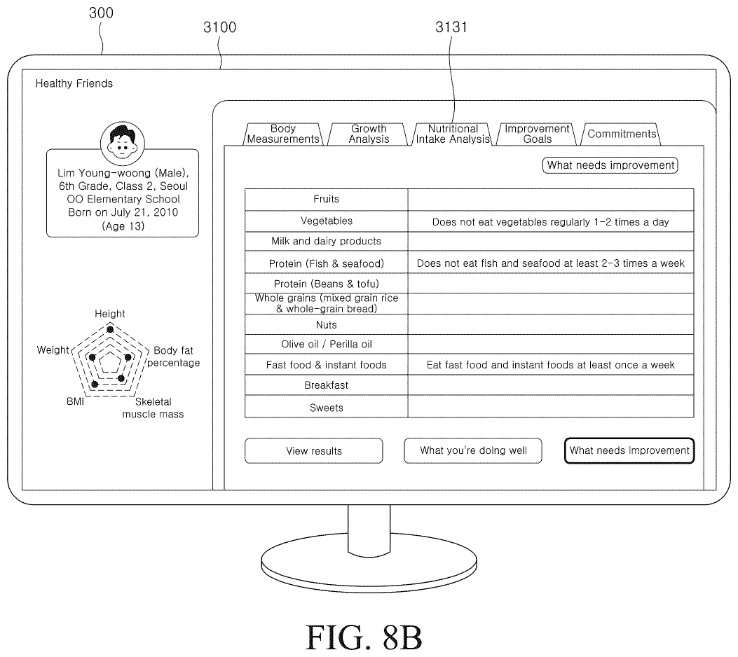

In addition, as shown in FIGS. 8A and 8B, a brief analysis result of the answer to the diet-related question may also be provided by being displayed in the nutrition intake analysis category 3131. In this case, the correct habits of the target object (corresponding to ‘doing well’ in the drawing) and the habits that need to be fixed (corresponding to ‘things to be fixed’ on the drawing) are respectively displayed so that the target object, guardian, and/or medical staff can easily visually check them.

Next, the processor 140 receives and obtains answer information about the target object generated based on the questionnaire provided by step S110 from the user terminal 200 (S120).

Next, the processor 140 inputs the answer information obtained by step S120 into a pre-trained artificial intelligence model and receives goal information based on the developmental state and nutritional state of the target object as an output (S130).

As described above, the goal information may include a target weight value, a target performance value, a target intake amount, and at least one educational material. In this case, the artificial intelligence model may calculate a score (hereinafter, referred to as “KIDMED score”) using the answer information based on the Mediterranean diet (hereinafter, referred to as “KIDMED”), and then generate goal information by using the calculated score to determine a value for each of the target weight value, the target performance value, and/or the target intake amount, and selecting at least one educational material corresponding to the target object from among a plurality of educational materials.

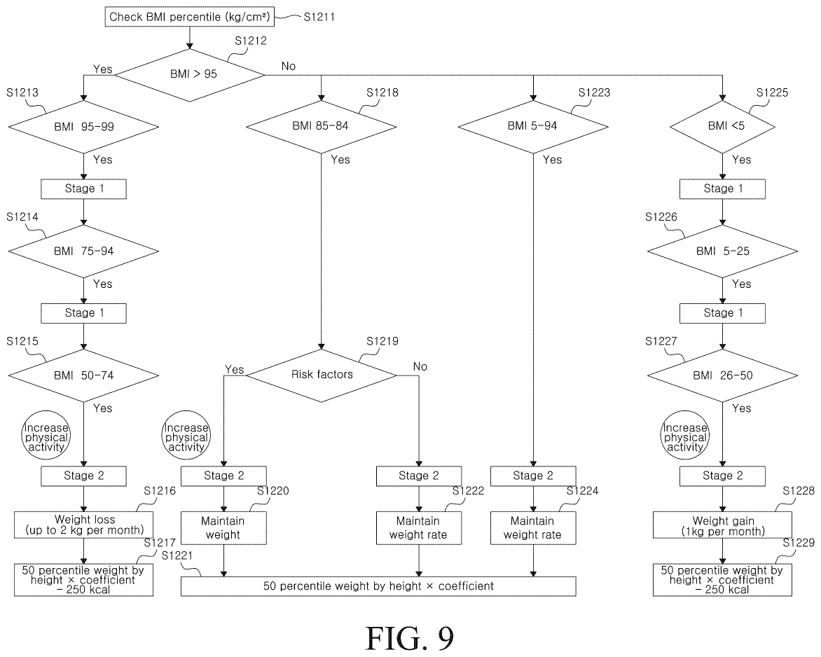

Among them, the target weight value may be determined based on a series of algorithms shown in FIG. 9. Based on this algorithm, weight control goals according to BMI percentiles are largely set, and final nutrition requirements can be calculated based on the set goals. At this time, the nutritional intervention method for weight control can be determined and applied according to each BIM percentile. That is, in the nutritional intervention method, a multiple nutritional intervention methods classified according to at least one of the BMI percentile (body mass index) and risk factors may be preset so that the target object may reach the target weight value. That is, it is possible to determine and provide a nutritional intervention method suitable for the target object by considering at least one of the BMI percentile and risk factors of the target object among a multiple nutritional intervention methods.

In FIG. 9, which will be described below, the nutritional intervention method is divided into stage 1 and stage 2 and applied. For example, stage 1 may indicate a method of setting the intake ratio of the three major nutrients, and stage 2 may indicate a method of setting the intake ratio of all nutrients according to the intake standard of the nutrients for Koreans.

Specifically, the BMI percentile of the target object is checked (S1211), and whether the BMI percentile exceeds 95 is determined (S1212).

As a result of the determination in step S1212, when the BMI percentile exceeds 95, it is determined again whether the BMI percentile falls within the range of 95 to 99 (S1213). If the BMI percentile falls within the range of 95 to 99, the first-stage nutritional intervention method is applied to calculate and provide the three major nutrient intake ratios corresponding to the range. Thereafter, it is determined whether the BMI percentile falls within the range of 75 to 94 at predetermined intervals (S1214), and if the BMI percentile falls within the range of 75 to 94, the nutrition intervention method of the first-stage is applied as it is, but the intake ratio of the three major nutrients corresponding to the range is calculated and provided. Thereafter, it is determined whether or not the BMI percentile falls within the range of 50 to 74 at each predetermined cycle (S1215), and if the BMI percentile falls within the range of 50 to 74, a second-stage nutritional intervention method is applied to calculate and provide an overall nutrient intake ratio corresponding to the range. Thereafter, a goal is determined for the weight loss of up to 2 kg per month and the increase amount of physical activity consumption of 250 kcal (S1216), and based on the determined goal, the weight of the 50 percentile by the height of the target object is multiplied by the coefficient, and then the calorie of the increased amount of physical activity, 250 kcal, is subtracted to calculate a final nutrient requirement (S1217).

Meanwhile, as a result of the determination in step S1212, when the BMI percentile is 95 or less, it is determined whether the BMI percentile falls within each preset range (S1218, S1223, S1225).

If, as a result of the determination of step S1218, if the BMI percentile falls within the range of 85 to 94, it is determined whether it is a risk factor (S1219), a second-stage nutritional intervention method is applied depending on whether it is a risk factor to determine each aiming at weight maintenance or weight-rate maintenance (S1220, S1222), based on each of the determined goals, the 50 percentile weight according to the height of the target object is multiplied by a coefficient to determine the final nutritional requirement (S1221).

In addition, if the BMI percentile falls within the range of 5 to 84 as a result of the determination of step S1223, the nutritional intervention method of stage 2 is applied to determine to maintain the weight rate (S1224), and based on the determined goal, the weight of the 50 percentile according to the height of the target object is multiplied by the coefficient to determine the final nutrient requirement (S1221).

On the other hand, if the BMI percentile is less than 5 as a result of the judgment in step S1225, the nutritional intervention method in stage 1 is applied to calculate and provide the intake ratio of the three major nutrients corresponding to the range. Thereafter, it is determined whether or not the BMI percentile falls within the range of 5 to 25 at each preset period (S1226), and if the BMI percentile falls within the range of 5 to 25, the nutritional intervention method of the stage 1 is applied as it is, but the three major nutrient intake ratios corresponding to the range are calculated and provided. Thereafter, it is determined whether the BMI percentile falls within the range of 26 to 50 at each predetermined cycle (S1227), and if the BMI percentile falls within the range of 26 to 50, a second-stage nutritional intervention method is applied to calculate and provide an overall nutrient intake ratio corresponding to the range. Thereafter, a goal is determined for a weight gain of up to 1 kg per month and a physical activity increase which further preserves 500 kcal (S1228), and a final nutritional requirement is calculated by multiplying the weight of the 50 percentile according to the height of the target object by the coefficient based on the determined goal, and then adding 500 kcal, which is the calorie of the physical activity increase, to calculate the final nutritional requirement (S1229).

In calculating the final nutritional requirement above, the weight percentile according to the height of the target object may be determined based on the weight of the 50 percentile according to the gender in Table 1 below.

TABLE 1

| Gender 1 | | | | | | Gender 2 | | | | | |
|---|---|---|---|---|---|---|---|---|---|---|---|
| | | | | weight | Standard | | | | | weight | Standard |
| Height | | | | percentile | score | Height | | | | percentile | score |
| (cm) | L | M | S | 50th | 0 | (cm) | L | M | S | 50th | 0 |
| 90 | 0.9511 | 13.1903 | 0.0749 | 13.2 | 13.2 | 88 | 0.114 | 12.335 | 0.080 | 12.3 | 12.3 |
| 91 | 0.8786 | 13.4411 | 0.0741 | 13.4 | 13.4 | 89 | 0.139 | 12.595 | 0.078 | 12.6 | 12.6 |
| 92 | 0.8111 | 13.6937 | 0.0734 | 13.7 | 13.7 | 90 | 0.164 | 12.854 | 0.077 | 12.9 | 12.9 |
| 93 | 0.7421 | 13.9462 | 0.0728 | 13.9 | 13.9 | 91 | 0.186 | 13.116 | 0.076 | 13.1 | 13.1 |
| 94 | 0.6772 | 14.2011 | 0.0722 | 14.2 | 14.2 | 92 | 0.209 | 13.378 | 0.075 | 13.4 | 13.4 |
| 95 | 0.6115 | 14.4558 | 0.0716 | 14.5 | 14.5 | 93 | 0.232 | 13.639 | 0.074 | 13.6 | 13.6 |
| 96 | 0.5449 | 14.7105 | 0.0711 | 14.7 | 14.7 | 94 | 0.234 | 13.899 | 0.074 | 13.9 | 13.9 |
| 97 | 0.4629 | 14.9664 | 0.0707 | 15.0 | 15.0 | 95 | 0.236 | 14.159 | 0.073 | 14.2 | 14.2 |
| 98 | 0.3809 | 15.2222 | 0.0702 | 15.2 | 15.2 | 96 | 0.238 | 14.419 | 0.072 | 14.4 | 14.4 |
| 99 | 0.2988 | 15.4779 | 0.0698 | 15.5 | 15.5 | 97 | 0.217 | 14.687 | 0.071 | 14.7 | 14.7 |
| 100 | 0.2576 | 15.7611 | 0.0702 | 15.8 | 15.8 | 98 | 0.198 | 14.955 | 0.071 | 15.0 | 15.0 |
| 101 | 0.2188 | 16.0443 | 0.0706 | 16.0 | 16.0 | 99 | 0.188 | 15.243 | 0.071 | 15.2 | 15.2 |
| 102 | 0.1823 | 16.3276 | 0.0709 | 16.3 | 16.3 | 100 | 0.179 | 15.531 | 0.072 | 15.5 | 15.5 |
| 103 | 0.1327 | 16.6311 | 0.0717 | 16.6 | 16.6 | 101 | 0.170 | 15.819 | 0.072 | 15.8 | 15.8 |
| 104 | 0.0869 | 16.9347 | 0.0724 | 16.9 | 16.9 | 102 | 0.122 | 16.122 | 0.073 | 16.1 | 16.1 |
| 105 | 0.0445 | 17.2382 | 0.0731 | 17.2 | 17.2 | 103 | 0.078 | 16.425 | 0.073 | 16.4 | 16.4 |
| 106 | −0.0295 | 17.5614 | 0.0740 | 17.6 | 17.6 | 104 | 0.036 | 16.728 | 0.074 | 16.7 | 16.7 |
| 107 | −0.0967 | 17.8847 | 0.0749 | 17.9 | 17.9 | 105 | −0.020 | 17.042 | 0.076 | 17.0 | 17.0 |
| 108 | −0.1580 | 18.2079 | 0.0758 | 18.2 | 18.2 | 106 | −0.071 | 17.357 | 0.077 | 17.4 | 17.4 |
| 109 | −0.2158 | 18.5519 | 0.0768 | 18.6 | 18.6 | 107 | −0.116 | 17.671 | 0.078 | 17.7 | 17.7 |
| 110 | −0.2682 | 18.8958 | 0.0778 | 18.9 | 18.9 | 108 | −0.187 | 18.006 | 0.080 | 18.0 | 18.0 |
| 111 | −0.3159 | 19.2397 | 0.0787 | 19.2 | 19.2 | 109 | −0.250 | 18.342 | 0.081 | 18.3 | 18.3 |
| 112 | −0.3718 | 19.6100 | 0.0799 | 19.6 | 19.6 | 110 | −0.332 | 18.691 | 0.083 | 18.7 | 18.7 |
| 113 | −0.4212 | 19.9804 | 0.0811 | 20.0 | 20.0 | 111 | −0.405 | 19.040 | 0.084 | 19.0 | 19.0 |
| 114 | −0.4652 | 20.3509 | 0.0822 | 20.4 | 20.4 | 112 | −0.469 | 19.390 | 0.085 | 19.4 | 19.4 |
| 115 | −0.4936 | 20.7471 | 0.0837 | 20.7 | 20.7 | 113 | −0.491 | 19.761 | 0.087 | 19.8 | 19.8 |
| 116 | −0.5175 | 21.1434 | 0.0851 | 21.1 | 21.1 | 114 | −0.510 | 20.133 | 0.088 | 20.1 | 20.1 |
| 117 | −0.5377 | 21.5398 | 0.0864 | 21.5 | 21.5 | 115 | −0.527 | 20.504 | 0.090 | 20.5 | 20.5 |
| 118 | −0.5359 | 21.9764 | 0.0882 | 22.0 | 22.0 | 116 | −0.535 | 20.911 | 0.091 | 20.9 | 20.9 |
| 119 | −0.5328 | 22.4131 | 0.0898 | 22.4 | 22.4 | 117 | −0.541 | 21.318 | 0.093 | 21.3 | 21.3 |
| 120 | −0.5287 | 22.8498 | 0.0914 | 22.8 | 22.8 | 118 | −0.545 | 21.725 | 0.094 | 21.7 | 21.7 |
| 121 | −0.5171 | 23.3392 | 0.0935 | 23.3 | 23.3 | 119 | −0.540 | 22.167 | 0.096 | 22.2 | 22.2 |
| 122 | −0.5052 | 23.8287 | 0.0956 | 23.8 | 23.8 | 120 | −0.534 | 22.609 | 0.098 | 22.6 | 22.6 |
| 123 | −0.4932 | 24.3183 | 0.0975 | 24.3 | 24.3 | 121 | −0.532 | 23.084 | 0.100 | 23.1 | 23.1 |
| 124 | −0.4634 | 24.8681 | 0.1000 | 24.9 | 24.9 | 122 | −0.528 | 23.559 | 0.101 | 23.6 | 23.6 |
| 125 | −0.4361 | 25.4179 | 0.1024 | 25.4 | 25.4 | 123 | −0.524 | 24.034 | 0.103 | 24.0 | 24.0 |
| 126 | −0.4108 | 25.9677 | 0.1047 | 26.0 | 26.0 | 124 | −0.533 | 24.542 | 0.105 | 24.5 | 24.5 |
| 127 | −0.3863 | 26.5725 | 0.1071 | 26.6 | 26.6 | 125 | −0.539 | 25.051 | 0.106 | 25.1 | 25.1 |
| 128 | −0.3636 | 27.1774 | 0.1094 | 27.2 | 27.2 | 126 | −0.539 | 25.595 | 0.108 | 25.6 | 25.6 |
| 129 | −0.3425 | 27.7822 | 0.1115 | 27.8 | 27.8 | 127 | −0.537 | 26.140 | 0.110 | 26.1 | 26.1 |
| 130 | −0.3098 | 28.4345 | 0.1139 | 28.4 | 28.4 | 128 | −0.535 | 26.684 | 0.112 | 26.7 | 26.7 |
| 131 | −0.2800 | 29.0867 | 0.1161 | 29.1 | 29.1 | 129 | −0.518 | 27.275 | 0.114 | 27.3 | 27.3 |
| 132 | −0.2529 | 29.7388 | 0.1183 | 29.7 | 29.7 | 130 | −0.502 | 27.866 | 0.116 | 27.9 | 27.9 |
| 133 | −0.2136 | 30.4329 | 0.1205 | 30.4 | 30.4 | 131 | −0.471 | 28.489 | 0.118 | 28.5 | 28.5 |
| 134 | −0.1782 | 31.1269 | 0.1226 | 31.1 | 31.1 | 132 | −0.443 | 29.113 | 0.120 | 29.1 | 29.1 |
| 135 | −0.1461 | 31.8208 | 0.1246 | 31.8 | 31.8 | 133 | −0.417 | 29.736 | 0.122 | 29.7 | 29.7 |
| 136 | −0.1128 | 32.5573 | 0.1265 | 32.6 | 32.6 | 134 | −0.373 | 30.385 | 0.123 | 30.4 | 30.4 |
| 137 | −0.0826 | 33.2937 | 0.1283 | 33.3 | 33.3 | 135 | −0.333 | 31.034 | 0.125 | 31.0 | 31.0 |
| 138 | −0.0550 | 34.0300 | 0.1301 | 34.0 | 34.0 | 136 | −0.296 | 31.683 | 0.126 | 31.7 | 31.7 |
| 139 | −0.0204 | 34.8059 | 0.1316 | 34.8 | 34.8 | 137 | −0.248 | 32.334 | 0.127 | 32.3 | 32.3 |
| 140 | 0.0113 | 35.5816 | 0.1331 | 35.6 | 35.6 | 138 | −0.203 | 32.985 | 0.128 | 33.0 | 33.0 |
| 141 | 0.0405 | 36.3573 | 0.1345 | 36.4 | 36.4 | 139 | −0.161 | 33.636 | 0.129 | 33.6 | 33.6 |
| 142 | 0.0682 | 37.1527 | 0.1354 | 37.2 | 37.2 | 140 | −0.121 | 34.336 | 0.130 | 34.3 | 34.3 |
| 143 | 0.0941 | 37.9481 | 0.1362 | 37.9 | 37.9 | 141 | −0.084 | 35.036 | 0.131 | 35.0 | 35.0 |
| 144 | 0.1184 | 38.7434 | 0.1371 | 38.7 | 38.7 | 142 | −0.050 | 35.831 | 0.132 | 35.8 | 35.8 |
| 145 | 0.1372 | 39.5638 | 0.1374 | 39.6 | 39.6 | 143 | −0.019 | 36.627 | 0.133 | 36.6 | 36.6 |
| 146 | 0.1551 | 40.3841 | 0.1378 | 40.4 | 40.4 | 144 | 0.011 | 37.422 | 0.133 | 37.4 | 37.4 |
| 147 | 0.1721 | 41.2044 | 0.1381 | 41.2 | 41.2 | 145 | 0.051 | 38.329 | 0.134 | 38.3 | 38.3 |
| 148 | 0.1733 | 42.0428 | 0.1382 | 42.0 | 42.0 | 146 | 0.089 | 39.236 | 0.134 | 39.2 | 39.2 |
| 149 | 0.1744 | 42.8813 | 0.1382 | 42.9 | 42.9 | 147 | 0.133 | 40.248 | 0.134 | 40.2 | 40.2 |
| 150 | 0.1755 | 43.7198 | 0.1383 | 43.7 | 43.7 | 148 | 0.175 | 41.262 | 0.134 | 41.3 | 41.3 |
| 151 | 0.1688 | 44.5648 | 0.1384 | 44.6 | 44.6 | 149 | 0.216 | 42.276 | 0.134 | 42.3 | 42.3 |
| 152 | 0.1625 | 45.4098 | 0.1385 | 45.4 | 45.4 | 150 | 0.248 | 43.324 | 0.133 | 43.3 | 43.3 |
| 153 | 0.1564 | 46.2547 | 0.1386 | 46.3 | 46.3 | 151 | 0.280 | 44.373 | 0.133 | 44.4 | 44.4 |
| 154 | 0.1457 | 47.0807 | 0.1383 | 47.1 | 47.1 | 152 | 0.299 | 45.378 | 0.131 | 45.4 | 45.4 |
| 155 | 0.1352 | 47.9066 | 0.1380 | 47.9 | 47.9 | 153 | 0.317 | 46.383 | 0.130 | 46.4 | 46.4 |
| 156 | 0.1251 | 48.7325 | 0.1377 | 48.7 | 48.7 | 154 | 0.336 | 47.389 | 0.129 | 47.4 | 47.4 |
| 157 | 0.1146 | 49.5276 | 0.1369 | 49.5 | 49.5 | 155 | 0.317 | 48.280 | 0.128 | 48.3 | 48.3 |
| 158 | 0.1042 | 50.3227 | 0.1362 | 50.3 | 50.3 | 156 | 0.298 | 49.171 | 0.127 | 49.2 | 49.2 |
| 159 | 0.0940 | 51.1177 | 0.1354 | 51.1 | 51.1 | 157 | 0.278 | 50.061 | 0.125 | 50.1 | 50.1 |
| 160 | 0.0898 | 51.9626 | 0.1344 | 52.0 | 52.0 | 158 | 0.250 | 50.852 | 0.125 | 50.9 | 50.9 |
| 161 | 0.0858 | 52.8075 | 0.1334 | 52.8 | 52.8 | 159 | 0.221 | 51.642 | 0.124 | 51.6 | 51.6 |
| 162 | 0.0819 | 53.6524 | 0.1324 | 53.7 | 53.7 | 160 | 0.193 | 52.432 | 0.123 | 52.4 | 52.4 |
| 163 | 0.0709 | 54.5408 | 0.1311 | 54.5 | 54.5 | 161 | 0.158 | 53.168 | 0.122 | 53.2 | 53.2 |
| 164 | 0.0599 | 55.4290 | 0.1298 | 55.4 | 55.4 | 162 | 0.123 | 53.904 | 0.122 | 53.9 | 53.9 |
| 165 | 0.0489 | 56.3173 | 0.1286 | 56.3 | 56.3 | 163 | 0.069 | 54.617 | 0.121 | 54.6 | 54.6 |
| 166 | 0.0378 | 57.2442 | 0.1275 | 57.2 | 57.2 | 164 | 0.017 | 55.331 | 0.121 | 55.3 | 55.3 |
| 167 | 0.0268 | 58.1710 | 0.1263 | 58.2 | 58.2 | 165 | −0.033 | 56.046 | 0.120 | 56.0 | 56.0 |
| 168 | 0.0159 | 59.0979 | 0.1252 | 59.1 | 59.1 | 166 | −0.088 | 56.809 | 0.120 | 56.8 | 56.8 |
| 169 | 0.0137 | 59.9961 | 0.1242 | 60.0 | 60.0 | 167 | −0.140 | 57.573 | 0.120 | 57.6 | 57.6 |
| 170 | 0.0117 | 60.8944 | 0.1232 | 60.9 | 60.9 | 168 | −0.194 | 58.340 | 0.120 | 58.3 | 58.3 |
| 171 | 0.0099 | 61.7927 | 0.1223 | 61.8 | 61.8 | 169 | −0.244 | 59.108 | 0.119 | 59.1 | 59.1 |
| 172 | 0.0040 | 62.5936 | 0.1214 | 62.6 | 62.6 | 170 | −0.293 | 59.878 | 0.119 | 59.9 | 59.9 |
| 173 | −0.0018 | 63.3945 | 0.1206 | 63.4 | 63.4 | 171 | −0.341 | 60.653 | 0.119 | 60.7 | 60.7 |
| 174 | −0.0076 | 64.1954 | 0.1199 | 64.2 | 64.2 | 172 | −0.386 | 61.430 | 0.119 | 61.4 | 61.4 |
| 175 | 0.0054 | 64.9942 | 0.1194 | 65.0 | 65.0 | | | | | | |
| 176 | 0.0187 | 65.7932 | 0.1189 | 65.8 | 65.8 | | | | | | |
| 177 | 0.0322 | 66.5927 | 0.1185 | 66.6 | 66.6 | | | | | | |
| 178 | 0.1207 | 67.4819 | 0.1185 | 67.5 | 67.5 | | | | | | |
| 179 | 0.2095 | 68.3739 | 0.1185 | 68.4 | 68.4 | | | | | | |
| 180 | 0.2987 | 69.2684 | 0.1184 | 69.3 | 69.3 | | | | | | |
| 181 | 0.3970 | 70.1667 | 0.1184 | 70.2 | 70.2 | | | | | | |
| 182 | 0.4947 | 71.0673 | 0.1184 | 71.1 | 71.1 | | | | | | |
| 183 | 0.5918 | 71.9701 | 0.1184 | 72.0 | 72.0 | | | | | | |
| 184 | 0.6982 | 72.8940 | 0.1183 | 72.9 | 72.9 | | | | | | |
| 185 | 0.8031 | 73.8193 | 0.1182 | 73.8 | 73.8 | | | | | | |
| 186 | 0.9063 | 74.7456 | 0.1181 | 74.7 | 74.7 | | | | | | |

That is, the calculation of the final nutrient requirement may be calculated by selecting the weight of the 50 percentile according to the height in Table 1 described above and multiplying the selected weight by the coefficient.

Furthermore, the coefficient may be determined based on the required energy coefficient per body weight according to age/gender in Table 2 below.

TABLE 2

| Age(international age) | Male | Female |
|---|---|---|
| 3- to 5-year-old | 80 | 80 |
| 6- to 8-year-old | 66 | 60 |
| 9- to 11-year-old | 54 | 49 |
| 12- to 14-year-old | 47 | 41 |
| 15- to 18-year-old | 42 | 37 |

Furthermore, in the case of the final nutritional requirement, it may be determined based on the proper ratio of childhood energy and the composition of daily nutrients in Table 3 below.

TABLE 3

| | Energy Composition Ratio | | | Daily Nutrient Composition | | |
|---|---|---|---|---|---|---|
| | Carbohydrate | | | Carbohydrate | | |
| Energy(kcal) | (%) | Protein(%) | Fat(%) | (g) | Protein (g) | Fat (g) |
| 1200 | 55(%) | 15(%) | 30(%) | 165 | 45 | 40 |
| 1300 | | | | 179 | 49 | 43 |
| 1400 | | | | 193 | 53 | 47 |
| 1500 | | | | 206 | 56 | 50 |
| 1600 | | | | 220 | 60 | 53 |
| 1700 | | | | 234 | 64 | 57 |
| 1800 | | | | 248 | 68 | 60 |
| 1900 | | | | 261 | 71 | 63 |
| 2000 | | | | 275 | 75 | 67 |
| 2100 | | | | 289 | 79 | 70 |
| 2200 | | | | 303 | 83 | 73 |
| 2300 | | | | 316 | 86 | 77 |
| 2400 | | | | 330 | 90 | 80 |
| 2500 | | | | 344 | 94 | 83 |

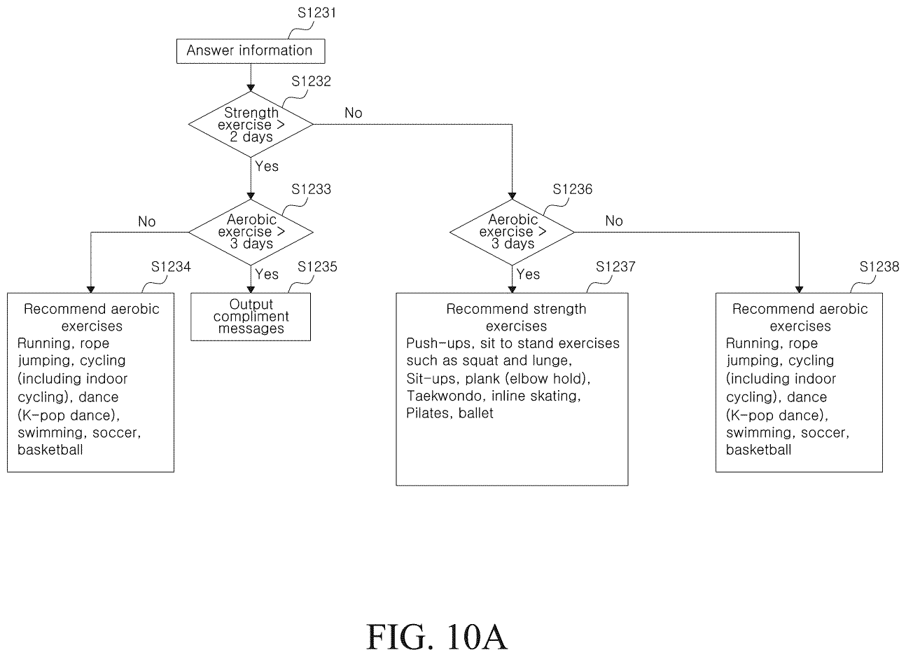

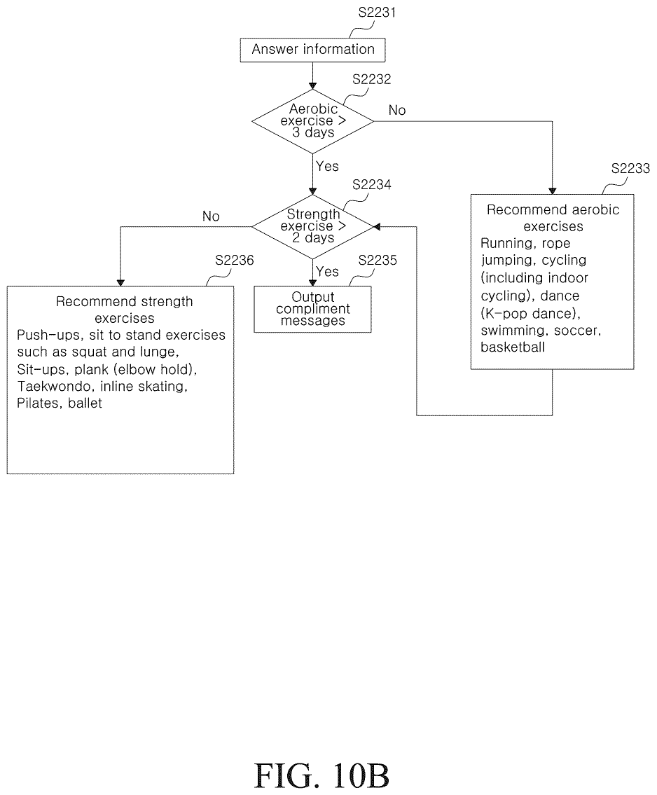

Meanwhile, a target performance value among them may be determined based on a series of algorithms shown in FIGS. 10A and 10B. Based on this algorithm, at least one exercise to be performed by the target object may be recommended as a recommended exercise set.

In this case, FIG. 10A illustrates a first embodiment in which the answer information related to the strength exercise is considered first, followed by consideration of the answer information related to the aerobic exercise, and FIG. 10B illustrates a second embodiment in which answer information related to the aerobic exercise is considered first, followed by consideration of the answer information related to the strength exercise.

First, referring to FIG. 10A, the processor 140 identifies an answer to an exercise-related question from the answer information obtained by step S120 (S1231), and determines whether the target object performs strength exercise for at least two days a week based on the answer (S1232). As a result of the determination in step S1232, when it is determined that the target object performs the strength exercise for at least two days a week, the processor 140 determines whether or not the target object performs the aerobic exercise for at least three days a week (S1233). If it is determined that the target object does not perform the aerobic exercise for at least three days a week, the processor 140 extracts at least one aerobic exercise from an exercise category classified as capable of being performed by the target object, among a plurality of exercises, to configure and provide a recommended exercise set. If it is determined that the target object performs the aerobic exercise for at least three days a week, the processor 140 outputs and provides a compliment message (S1235).

Meanwhile, as a result of the determination in step S1232, when it is determined that the target object does not perform the strength exercise for at least two days a week, the processor 140 determines whether or not the aerobic exercise is performed for at least three days a week (S1236). If it is determined that the target object performs aerobic exercise for at least three days a week, the processor 140 extracts at least one strength exercise from an exercise category classified as capable of being performed by the target object among a plurality of exercises to configure and provide a recommended exercise set (S1237). If it is determined that the target object does not perform the aerobic exercise for at least three days a week, the processor 140 extracts at least one aerobic exercise from an exercise category classified as capable of being performed by the target object among a plurality of exercises to configure and provide a recommended exercise set (S1238).

Meanwhile, referring to FIG. 10B, the processor 140 identifies an answer to the exercise-related question from the answer information obtained by step S120 (S2231), and determines whether the target object performs the aerobic exercise for at least three days a week based on the answer (S2232).

As a result of the determination in step S2232, when it is determined that the target object does not perform the aerobic exercise for at least three days a week, the processor 140 extracts at least one aerobic exercise from an exercise category classified as being able to be performed by the target object among a plurality of exercises to configure and provide a recommended exercise set (S2233).

Meanwhile, as a result of the determination in step S2232, if it is determined that the aerobic exercise is performed for three days or more a week, the processor 140 determines whether the target object performs the strength exercise for two days or more a week (S2234). In addition, the processor 140 proceeds to step S2234 in a state in which the recommended exercise set is provided by step S2233 to further determine whether the target object performs strength exercise for at least two days a week (S2234).

As a result of the determination in step S2234, when it is determined that the target object performs strength exercise for at least two days a week, the processor 140 outputs and provides a compliment message (S2235).

If, as a result of the determination in step S2234, it is determined that the target object does not perform the strength exercise for two or more days a week, the processor 140 extracts at least one strength exercise from an exercise category classified as being able of being performed by the target object among a plurality of exercises to configure and provide a recommended exercise set (S2236).

The recommended exercise set illustrated in FIGS. 10A and 10B is only an embodiment, and some exercises include therein may be excluded, or other exercises may be added. Further, as an example, in an answer to a question related to exercise of the target object, a recommended exercise set may be edited based on strength exercise and/or aerobic exercise that the target object is already performing, or the recommended exercise set may be edited based on strength exercise and/or aerobic exercise that the target object wants to do, that the target object is interested in. Here, the aerobic exercise may include at least one of running, rope jumping, cycling (including indoor and outdoor cycling), dancing, badminton, swimming, soccer, basketball, trampoline, and strength exercise may include at least one of push-up, sit to stand exercises such as squat and lunge, sit-up, plank (holding up with elbows), Taekwondo, inline skating, skateboarding, pilates, ballet, kids flying yoga, kids bungy physio, and climbing. However, the types of exercise included in aerobic exercise and/or strength exercise are not limited to those listed above, and some of them may be excluded or other events may be added.

In addition, although not shown in FIGS. 10A and 10B, the number of times of performing each exercise included in the recommended exercise set may be determined and provided, and the number of times of performing each exercise may be determined based on the age or body data of the target object. To this end, the service server 100 may have predetermined and stored the number of times of each exercise for each age of the target object. However, since there may be cases where the target object is physically more developed than the age, the processor 140 may determine the number of each exercise corresponding to the age based on the body data by flexibly adjusting the number of each exercise corresponding to the age.

In addition, an algorithm for determining target intake amount among them is not illustrated in a separate drawing, but the processor 140 can figure out the intake problem of the target object based on a pre-stored intake reference table, determine at least one nutrient that the target object needs to consume and an intake ratio of each nutrient, and then determine a target intake amount for at least one of food and supplements containing each nutrient.

Next, the processor 140 sets up at least one mission for the target object on the platform using the goal information provided by step S130. That is, the processor 140 sets the target weight value, target performance value, and target intake amount included in the goal information and provides them as a mission so that the target object and/or guardian can visually confirm them, thereby enabling them to be achieved.

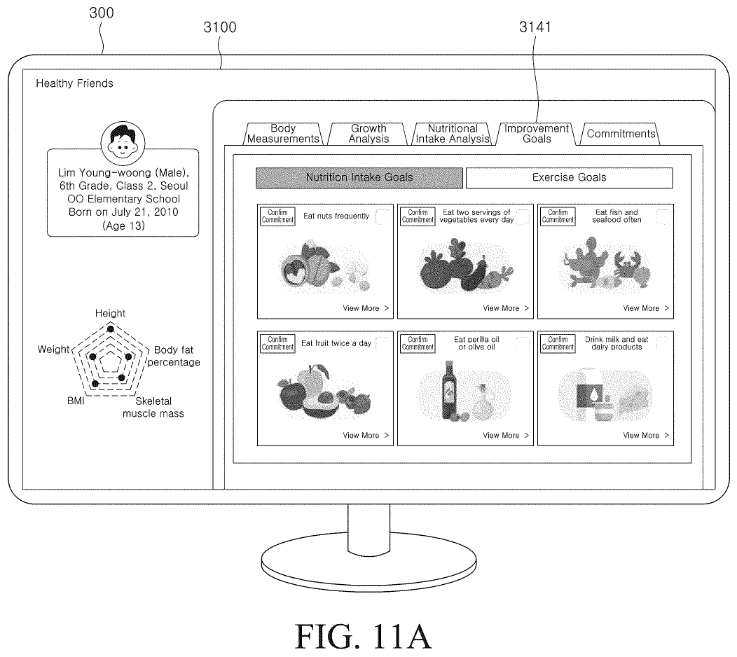

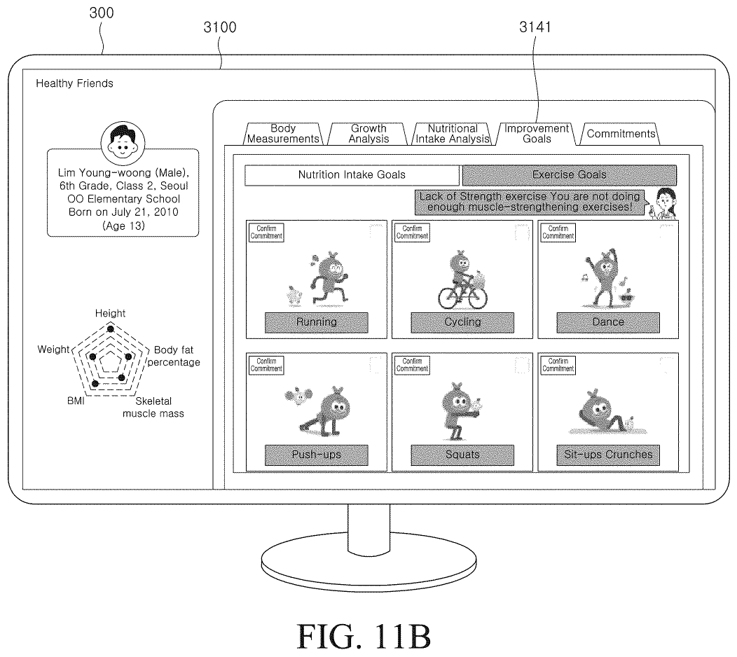

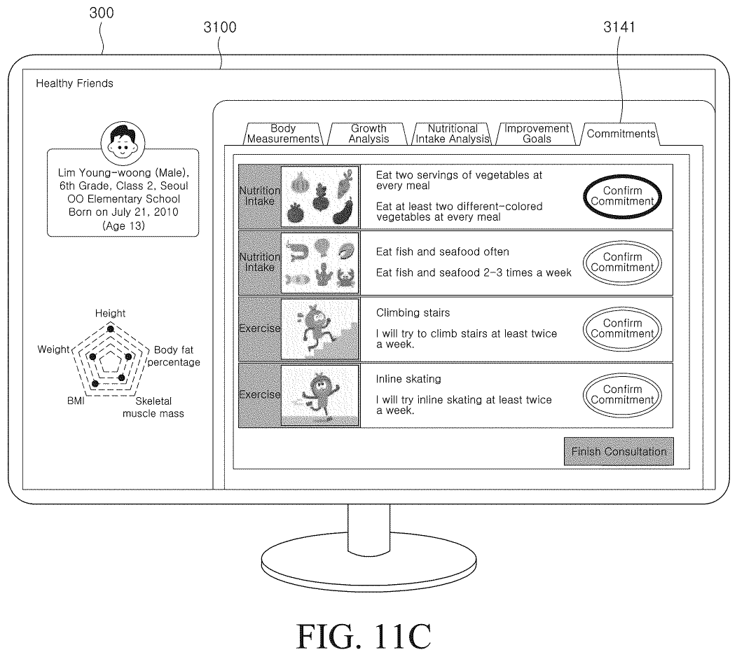

Specifically, as illustrated in FIGS. 11A to 11B, a nutrition intake goal and an exercise goal may be set and provided in the improvement target category 3141, respectively, based on the goal information.

In this case, the nutrition intake goal and the exercise goal may be displayed in at least one manner using a separate character, an icon, or the like, or a character generated based on an image of the target object to induce a visual interest of the target object. In this case, the target object may be configured to click a separately generated appointment button to remind each goal.

Meanwhile, as illustrated in FIG. 11C, by listing each goal that the target object must perform in the commitment category 3151 in a list format and having the target object commitment to perform the goal by selecting/touching a separately configured button or writing along with the text, motivation for goal performance can be provided.

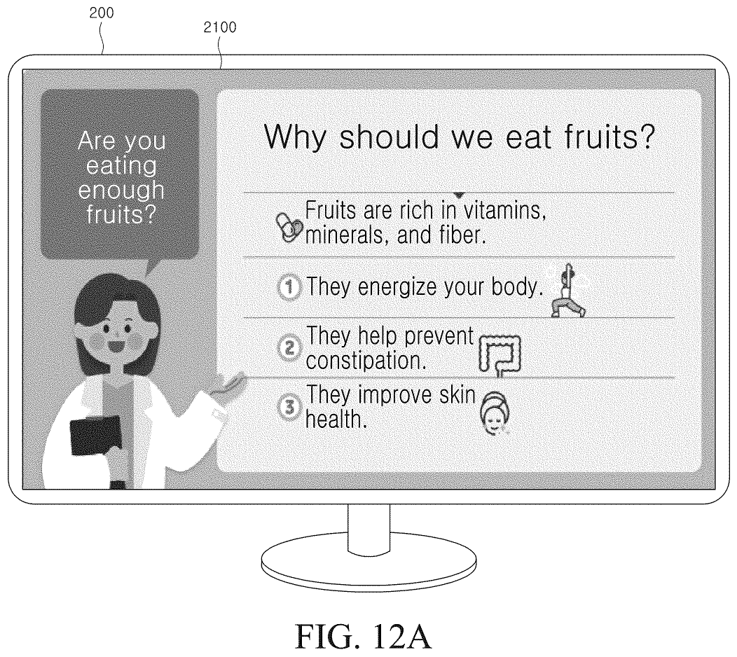

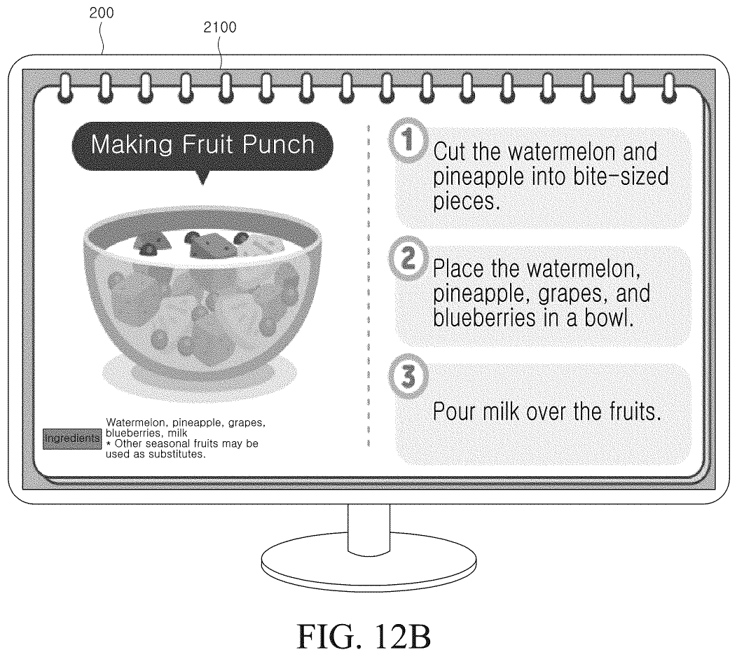

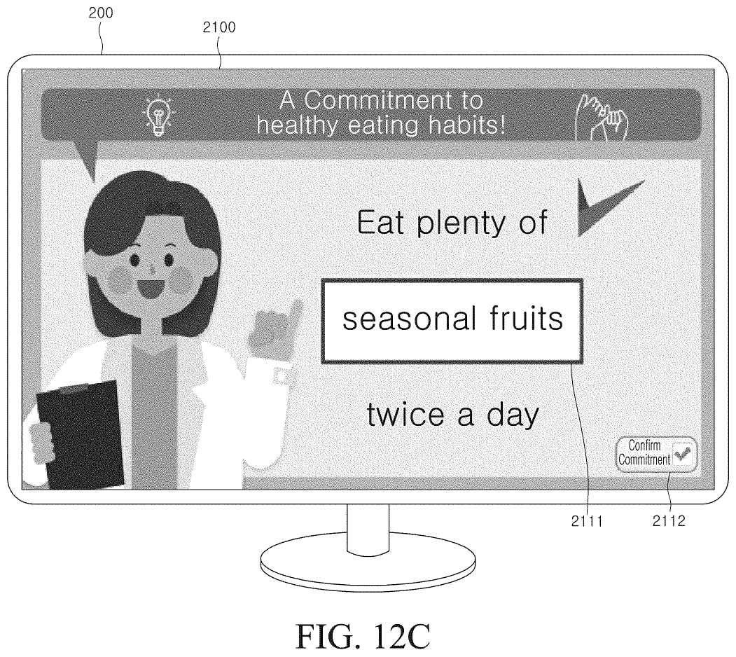

In addition, since the goal information may include at least one educational material, educational materials corresponding thereto may be attached to each goal. For example, as shown in FIGS. 12A to 12C, various contents such as why fruits should be consumed, how much fruits should be consumed, and what fruits should be consumed according to the season may be included in the educational material attached to the goal related to fruit intake so that the target object can understand the fundamental reason for why the goal should be performed. In addition, as in FIG. 12B, a recipe can be included on how to use and consume the fruit, and as in FIG. 12C, after the target object completes all training, the target object can be instructed to write down the text displayed in the text window 2111 so that the target object can recall the content of the training, or select/touch a separately configured commitment button 2112. In this case, the at least one educational material may be provided in the form of at least one of a video, a voice, a document, an image, and a link, but the form is not limited thereto.

Next, when the input information about the target object is received, the processor 140 determines whether or not to perform at least one mission set by step S140 based on the input information, that is, monitors (S150).

In this case, the input information may be input by uploading various information and/or data for proving that the target object has performed the goal by selecting/touching each goal set on the platform.

This determination operation in step S150 may be performed whenever the target object performs each mission included in at least one mission, and when input information for a specific mission is input from the user terminal 200, whether the mission is performed is determined based on whether the input information satisfies set value of the corresponding mission (corresponding to a target value determined through the goal information).

Although not shown in FIG. 4, the processor 140 may output a notification according to the determination result of step S150, that is, the monitoring result.

If it is determined in step S150 that the execution value of the target object included in the input information does not satisfy the set value of the corresponding mission, since the target object has not performed the mission, a pre-stored encouraging message is output and provided, and if it is determined that the execution value of the target object included in the input information satisfies the set value of the corresponding mission, since the target object has performed the mission, a pre-stored compliment message may be output and provided.

However, this is only an embodiment, and the words of encouraging message and compliment message may be newly created according to the situation of the target object, or may be written by a guardian or medical staff, but are not limited thereto.

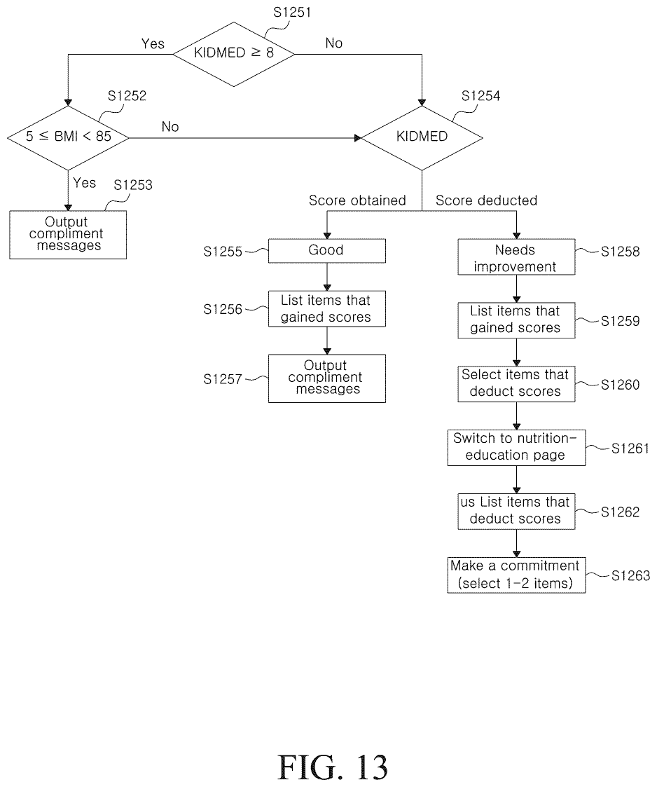

In this case, the processor 140 may output and provide an encouraging message and/or a compliment message in the form of at least one of a pop-up window, a voice message, and a text. Meanwhile, as an embodiment, as shown in FIG. 13, an algorithm for recalculating and updating the KIDMED score may be further applied depending on whether the target object has reached a developmental state and/or a nutritional state.

Specifically, after the target object performs all missions, the processor 140 compares the KIDMED score of the target object with a preset threshold (score information) (e.g., 8) (S1251), and if the KIDMED score is greater than or equal to the threshold as a result of the comparison, determines whether the BMI percentile of the target object falls within the range of 5 to 85 (S1252). Here, the KIMED score of the target object may be calculated based on the questionnaire by the artificial intelligence model in step S130.

If, as a result of the determination of S1252, if the BMI percentile of the target object falls within the range of 5 to 85, the processor 140 outputs and provides a compliment message (S1253), and if the BMI percentile of the target object does not fall within the range of 5 to 85, it is determined whether to obtain or subtract the score based on the KIDMED score (S1254).

If it is determined that the state is good and it is determined that the KIDMED score is obtained (S1255), the processor 140 lists the score acquisition item (S1256), and then outputs and provides a compliment message (S1257).

Further, when it is determined that the state needs to be improved and it is determined that the KIDMED score is deducted (S1258), the processor 140 lists the score deduction items (S1259), and then selects the score deduction item (S1260). Thereafter, the processor 140 switches to the nutrition education page corresponding to the score deduction item selected by step S1260 (S1261), lists the score deduction item (S1262), and performs an appointment for the score deduction item from the target object (S1263).

Meanwhile, although not illustrated in FIG. 4, after step S130, an analysis result of analyzing the answer information of the target object, that is, the analysis information, may be provided to the guardian terminal 500. In this case, the analysis information may include each data included in the goal information.

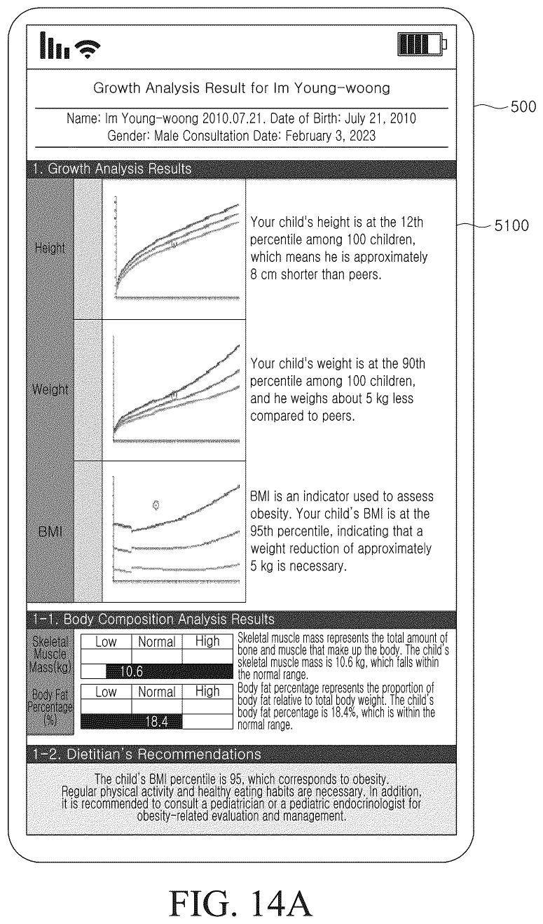

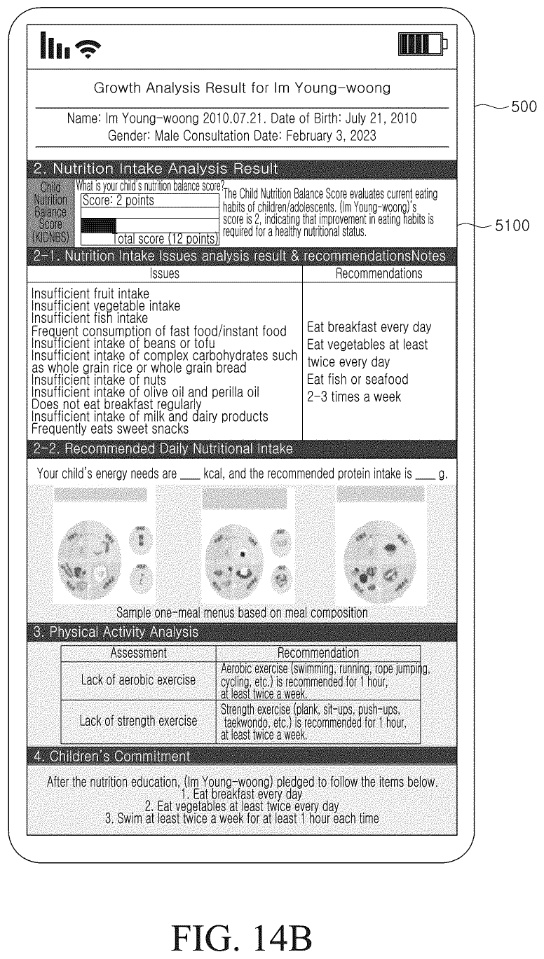

FIGS. 14A to 14B illustrate an example of analysis information provided to the guardian terminal 500, and largely include an analysis result of growth of a target object, an analysis result of body composition, an analysis result of nutrient intake, and an analysis result of physical activity, and the findings of medical staff may be described in each analysis result. At this time, the results of each analysis may be displayed to facilitate visual confirmation in the form of not only text, but also a graph, etc., and the results of the comparison with the peers of the target object may allow the guardian to easily understand the degree of development of the target object relative to the peers.

Additionally, the processor 140 may include and provide recommendations in the analysis information to remind the target object of the recommendations contained in the goal information, thereby allowing the target object to input the recommendations based on the target object's voice or text. In this case, the processor 140 may store and manage the recommendations as a response to the analysis information when the target object inputs the voice or text.

Therefore, the present disclosure provides a mission to a child, which, when achieved, provides rewards such as scores, and induces interest in the child through visual and auditory effects, thereby motivating the child to perform the mission, thereby enabling voluntary participation in health care activities.

Although the embodiments of the present invention have been described in more detail with reference to the attached drawings, the present invention is not necessarily limited to these embodiments, and various modifications can be made without departing from the technical idea of the present invention. Accordingly, the embodiments disclosed in the present invention are not intended to limit the technical idea of the present invention, but to explain it, and the scope of the technical idea of the present invention is not limited by these embodiments. Therefore, it should be understood that the embodiments described above are exemplary in all aspects and not restrictive. The protection scope of the present invention should be interpreted by the claims below, and all technical ideas within the equivalent scope should be interpreted as being included in the scope of the rights of the present invention.

What is claimed is:

1. A method for platform-based growth and dietary management support services for children and adolescents, performed by an apparatus, comprising:
providing a questionnaire including a plurality of questions each related to body, diet, and exercise to a target object;
obtaining answer information based on the questionnaire from the target object;
receiving goal information according to a developmental status and nutritional status of the target object as output based on a score calculated according to the Mediterranean diet by inputting the answer information into a pre-trained artificial intelligence model;
setting at least one mission for the target object on the platform using the goal information; and
monitoring, when input information for the target object is received, whether to perform the at least one mission based on the input information.

2. The method of claim 1,
the answer information is obtained based on a questionnaire mapped to the age of the target object and body data measured by a pre-connected measuring terminal,
the questionnaire is divided into physical items, diet items, and exercise items, and is configured to include at least one questionnaire in each item, and
the body data includes a measurement for at least one of weight, height, blood sugar, blood pressure, pulse, body fat, oxygen saturation, body temperature, heart rate, respiration rate, and electrocardiogram.

3. The method of claim 1,
the artificial intelligence model calculates a KIDMED score using the answer information based on the Mediterranean diet and generates the goal information including a target weight value, a target performance value, a target intake amount, and at least one educational material using the calculated score.

4. The method of claim 3,
the artificial intelligence model, when generating the goal information, determines the target weight value based on at least one of the body mass index and risk factors of the target object, identifies recommended exercise items corresponding to the type and number of exercises performed by the target object, determines the target performance value for each exercise included in the recommended exercise items by considering the physical information of the target object, determines at least one nutrient that needs to be consumed and the intake ratio of each nutrient by identifying the intake problem of the target object, and determines the target intake amount for at least one of the foods and supplements containing each nutrient.

5. The method of claim 3,
the at least one mission includes at least one of whether training has been completed based on the at least one educational material, whether the target performance value for each exercise has been achieved, and whether the target intake amount for at least one of food and supplements containing each nutrient has been achieved.

6. The method of claim 4,
the artificial intelligence model, when determining the target weight value, determines and provides any one of the multiple nutritional intervention methods by considering at least one of the body mass index and risk factors so as to reach the target weight value.

7. The method of claim 6,
the multiple nutritional intervention methods includes a method of setting an intake ratio of three major nutrients and a method of setting an intake ratio of all nutrients.

8. The method of claim 3,
the KIDMED score is determined by automatically adding or subtracting a preset score for a positive or negative answer for each questionnaire question.

9. The method of claim 8, further comprising:
adding or subtracting the KIDMED score of the target object according to whether to perform the at least one mission based on preset score information.

10. The method of claim 1, further comprising:
according to a result of monitoring whether to perform the at least one mission, generating and providing a notification including an encouraging message or a compliment message in the form of at least one of a pop-up window, a voice message, and a text.

11. An apparatus for platform-based growth and dietary management support services for children and adolescents, comprising:
a communication interface;
a memory; and
a processor operably connected with the communication interface and the memory,
the processor being configured to:
provide a questionnaire including a plurality of questions each related to body, diet, and exercise to a target object;
obtain answer information based on the questionnaire from the target object;
receive goal information based on the developmental status and nutritional status of the target object as output based on a score calculated according to the Mediterranean diet by inputting the answer information into a pre-trained artificial intelligence model;
set at least one mission for the target object on the platform using the goal information; and
monitor, when input information for the target object is received, whether to perform the at least one mission based on the input information.

12. The apparatus of claim 11,
the answer information is obtained based on a questionnaire mapped to the age of the target object and body data measured by a pre-connected measuring terminal,
the questionnaire is divided into physical items, diet items, and exercise items, and is configured to include at least one questionnaire in each item, and
the body data includes a measurement for at least one of weight, height, blood sugar, blood pressure, pulse, body fat, oxygen saturation, body temperature, heart rate, respiration rate, and electrocardiogram.

13. The apparatus of claim 11,
the artificial intelligence model calculates a KIDMED score using the answer information based on the Mediterranean diet and generates the goal information including a target weight value, a target performance value, a target intake amount, and at least one educational material using the calculated score.

14. The apparatus of claim 13,
the processor, when generating the goal information,
determines the target weight value based on at least one of the body mass index and risk factors of the target object,
identifies recommended exercise items corresponding to the type and number of exercises performed by the target object,
determines the target performance value for each exercise included in the recommended exercise items by considering the physical information of the target object,
determines at least one nutrient that needs to be consumed and the intake ratio of each nutrient by identifying the intake problem of the target object, and
determines the target intake amount for at least one of the foods and supplements containing each nutrient.

15. The apparatus of claim 13,
the at least one mission includes at least one of whether training has been completed based on the at least one educational material, whether the target performance value for each exercise has been achieved, and whether the target intake amount for at least one of food and supplements containing each nutrient has been achieved.

16. The apparatus of claim 14,
the processor, when determining the target weight value, further determine and provide any one of the multiple nutritional intervention methods in consideration of at least one of the body mass index and risk factors so as to reach the target weight value.

17. The apparatus of claim 16,
the multiple nutritional intervention methods includes a method of setting an intake ratio of three major nutrients and a method of setting an intake ratio of all nutrients.

18. The apparatus of claim 13,
the KIDMED score is determined by automatically adding or subtracting a preset score for a positive or negative answer for each questionnaire question.

19. The apparatus of claim 18,
the processor is configured to add or subtract the KIDMED score of the target object depending on whether the target object performs the at least one mission based on preset score information.

20. The apparatus of claim 11,
the processor is configured to generate and provide a notification including an encouraging message or a compliment message in the form of at least one of a pop-up window, a voice message, and text, according to a result of monitoring whether to perform the at least one mission.
